(19) United States
(12) Patent Application Publication
(10) Pub. No.: US 2026/0104329 A1
(43) Pub. Date: Apr. 16, 2026

(54) COMPOSITIONS, SYSTEMS, AND METHODS FOR MULTIPLE ANALYSES OF CELLS

(71) Applicant: Deepcell, Inc., Menlo Park, CA (US)

(72) Inventors: Mahdokht MASAELI, Los Altos, CA (US); Mahyar SALEK, Los Altos, CA (US)

(21) Appl. No.: 19/217,464

(22) Filed: May 23, 2025

Related U.S. Application Data
(63) Continuation of application No. 18/926,648, filed on May 23, 2025.
Application No. 18/926,648 is a continuation of application No. PCT/US23/19966, filed on Apr. 26, 2023.
(60) Provisional application No. 63/335,300, filed on Apr. 27, 2022.

(51) Int. Cl. G01N 1/30; G01N 15/10; G01N 15/1404; G01N 33/533; G01N 33/536; G01N 33/569

(52) U.S. Cl. CPC G01N 1/30; G01N 15/1404; G01N 33/533; G01N 33/536; G01N 33/569; G01N 2015/1006

(57) ABSTRACT

The present disclosure provides systems, methods, and compositions for cell analysis and/or partitioning. In an aspect, the present disclosure provides a method of imaging a cell. The method can comprise: (a) staining the cell using at least one dye, (b) rotating the cell in a field of view of an imaging device, and (c) imaging the cell to create a cell image.

### CROSS-REFERENCE

This application is a continuation of U.S. application Ser. No. 18/926,648, filed Oct. 25, 2024, which is a continuation of International Patent Application No. PCT/US23/19966, filed Apr. 26, 2023, which claims the benefit of U.S. Provisional Ser. No. 63/335,300 , filed Apr. 27, 2022, each of which is incorporated herein by reference in its entirety.

### BACKGROUND

Analysis of a cell (e.g., determination of a type or a state of the cell) can be accomplished by examining, for example, one or more images of the cell or sequencing data of the cell (e.g., gene fragment analysis, whole-genome sequencing, whole-exome sequencing, RNA-seq, etc.). Such methods can be used to identify cell type (e.g., stem cell or differentiated cell) or cell state (e.g., healthy or disease state). Such methods can require treatment of the cell (e.g., antibody staining, cell lysis or sequencing, etc.) that can be time-consuming and/or costly.

### SUMMARY

In view of the foregoing, recognized herein is a need for alternative compositions, systems, and methods for analyzing cells (e.g., previously uncharacterized or unknown cells).

Recognized herein is a need for compositions, systems, and methods for imaging and analyzing cells, such as cells that are tagged (e.g., stained with a polypeptide, such as an antibody, against a target protein of interest within the cell; with a polynucleotide against a target gene of interest within the cell; with probes to analyze gene expression profile of the cell via polymerase chain reaction; or with a small molecule substrate that is modified by the target protein).

Also recognized herein is a need for compositions, systems, and methods for analyzing and sorting (or partitioning) cells (e.g., with or without a tag) that are characterized to exhibit one or more characteristics of interest (e.g., image characteristics based on one or more images of each cell). In some embodiments, a tag can be a heterologous marker to a cell, and one or more components (e.g., membrane, proteins, polynucleotide sequence, etc.) of the cells can be tagged with the heterologous marker prior to, simultaneously with, or subsequent to imaging. In some embodiments, one or more components of the cells can be tagged with the heterologous marker prior to, simultaneously with, or subsequent to sorting (or partitioning into one or more chambers).

Further recognized herein is a need for compositions, systems, and methods for subjecting cells to a plurality of analysis modes, e.g., (i) imaging and (ii) one or more omics (e.g., genomics, transcriptomics, proteomics, or metabolomics).

In an aspect, the present disclosure provides a method of imaging a cell, the method comprising: staining the cell using at least one dye; rotating the cell in a field of view of an imaging device; and imaging the cell to create a cell image.

In another aspect, the present disclosure provides a method comprising: contacting a first cell population to a dye that distinguishes a dye target feature of a subset of the first cell population; imaging the first cell population; identifying an image feature of the first population that correlates to dye binding; imaging a second cell population; and sorting the second cell population based upon presence of the image characteristic.

In another aspect, the present disclosure provides a method of producing a population enriched for imaged cells sharing a common image characteristic, the method comprising: imaging a cell to obtain a cell image; comparing the cell image to a database; delivering the cell to a partition comprising a plurality of cells at least some of which have an image characteristic similar to an independent image of the database; and correlating the partition to the cell image.

In another aspect, the present disclosure provides a method of cell sorting, comprising: generating a first image of a first cell of a population of cells; delivering the first cell to a first partition; generating a second image of a second cell of the population of cells, wherein the second image is similar to the first image; and delivering the second cell to the first partition.

In another aspect, the present disclosure provides a method of tracking an imaged cell, the method comprising: imaging a cell to generate an imaged cell; delivering the imaged cell to a partition; and associating an image of the imaged cell with the partition.

In another aspect, the present disclosure provides a reservoir comprising a plurality of individually imaged cells, wherein an individually imaged cell of the plurality is labeled with a heterologous marker.

In another aspect, the present disclosure provides an emulsion comprising a plurality of aqueous partitions in an oil carrier held in a single well, wherein at least some of the aqueous partitions comprise one individually imaged cell per partition.

In another aspect, the present disclosure provides a method of tracking an imaged cell, the method comprising: imaging a cell; and delivering the imaged cell and a marker to a common partition.

In another aspect, the present disclosure provides a method of tracking an imaged cell, comprising: providing surface comprising a plurality of marker spots; imaging a cell; and delivering the imaged cell to a marked spot of the plurality of barcode oligo spots.

In another aspect, the present disclosure provides a method of assigning characteristics to subpopulations of a cell population, the method comprising measuring a phenotypic characteristic of at least some cells of the cell population; assigning the at least some cells of the cell population to one subpopulation of at least two subpopulations; and correlating the one subpopulation of at least two subpopulations to an independently expected subpopulation.

In another aspect, the present disclosure provides a method of attributing characteristics to subpopulations of a cell population, comprising assigning cells of a cell population into a plurality of subpopulations; correlating the plurality of subpopulations to a cell population dataset comprising dataset subpopulations having known characteristics, and attributing the known characteristics to subpopulations of the plurality of subpopulations.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications, and NCBI accession numbers mentioned in this specification are herein incorporated by reference to the same extent as if each individual publication, patent, patent application, or NCBI accession number was specifically and individually indicated to be incorporated by reference. To the extent publications and patents, patent applications, or NCBI accession numbers incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure”and “FIG.”herein), of which:

FIG. 1 schematically illustrates an example method for classifying a cell.

FIG. 2 schematically illustrates a cell morphological analysis platform operatively coupled to a single cell analysis module.

FIG. 3 schematically illustrates a cell morphological analysis platform operatively coupled to a multi-well plate or a microarray for single cell analysis.

FIG. 4A schematically illustrates use of a microarray spot with a nucleic acid barcode for nucleic acid sequencing.

FIG. 4B schematically illustrates use of emulsion with nucleic acid barcode for nucleic acid sequencing.

FIG. 5 schematically illustrates a cell analysis platform for analyzing image data of one or more cells.

FIGS. 6A-6B schematically illustrates an example microfluidic system for sorting one or more cells.

FIG. 7 shows a computer system that is programmed or otherwise configured to implement methods provided herein.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Unless defined otherwise, all technical and scientific terms used herein have the same meaning as commonly understood by one of ordinary skill in the art to which the present disclosure belongs. In case of conflict, the present application including the definitions will control. Also, unless otherwise required by context, singular terms shall include pluralities and plural terms shall include the singular.

The term “heterologous marker” as used herein generally refers to a heterologous composition detectable by one or more analytical or sensing techniques, such as, for example, fluorescence detection, spectroscopic detection, photochemical detection, biochemical detection, immunochemical detection, electrical detection, optical detection, chemical detection, or omics (e.g., genomics, transcriptomics, or proteomics, etc.). The heterologous marker can be a tag that can be coupled to (e.g., covalently or non-covalently) at least a portion of a cell, such as a cellular component.

In some embodiments, the heterologous marker can exhibit specific binding affinity to the at least the portion of the cell. The heterologous marker can be a label or an identifier that can convey information about the at least the portion of the cell (e.g., an analyte derived from the cell, or the cell in its entirety). The heterologous marker can be, for example, a polypeptide (e.g., an antibody or a fragment thereof), a nucleic acid molecule (e.g., a deoxyribonucleic acid (DNA)molecule, a ribonucleic acid (RNA) molecule, etc.) exhibiting at least a partial complementarity to a target nucleic acid sequence of the cell, or a small molecule configured to bind to a target epitope (e.g., a polypeptide sequence, a polynucleotide sequence, one or more polysaccharide moieties) of the cell.

In some embodiments, the heterologous marker can be a unique barcode. Barcodes as disclosed herein can have a variety of different formats. Non-limiting examples of a barcode can include polynucleotide barcodes, random nucleic acid and/or amino acid sequences, and synthetic nucleic acid and/or amino acid sequences. A barcode can be attached to the at least the portion of the cell (e.g., a nucleic acid sequence derived from the cell) in a reversible or irreversible manner. A barcode can be added to, for example, a fragment of a DNA or RNA sample derived from the cell, before, during, and/or after characterization of the cell (e.g., imaging of the cell, sequencing of the cell, etc.). In some cases, barcodes can allow for identification and/or quantification of different cells within a cell population. In some cases, barcodes can allow for identification and/or quantification of individual sequencing-reads.

In some embodiments, the heterologous marker can be an optically detectable moiety, such as a dye. In some embodiments, the heterologous marker can comprise or can be functionalized with (e.g., covalently or non-covalently) one or more optically detectable moieties, such as, a dye (e.g., tetramethylrhodamine isothiocyanate (TRITC), Quantum Dots, CY3 and CY5), biotin-streptavidin conjugates, magnetic beads, fluorescent dyes (e.g., fluorescein, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads.

The term “cellular component” as used herein generally refers to matter derived from a cell, such as matter contained inside a cell (i.e., intracellular) or presented outside of the cell (i.e., extracellular). A cellular component can include matter naturally derived from the cell (e.g., from the membrane of the cell, from the interior of the cell, components secretable or secreted by the cell, etc.) as well as originally foreign agents (e.g., microorganisms, viruses, asbestos, or compounds or extracellular origin) that exist inside the cell. Non-limiting examples of a cellular component can include an amino acid, a polypeptide (e.g., a peptide fragment, a protein, etc.), ion (e.g., Na+, Mg+, Cu+, Cu2+, Zn2+, Mn2+, Fe2+, and Co2+), polysaccharides, lipid (e.g., fats, waxes, sterols, fat-soluble vitamins such as vitamins A, D, E, and K, monoglycerides, diglycerides, triglycerides, or phospholipids), a nucleotide, a polynucleotide (e.g., DNA or RNA), particle (e.g., nanoparticle), fibers (e.g., asbestos fibers), cytoplasm, organelle (e.g., mitochondria, peroxisome, plastid, endoplasmic reticulum, flagellum, Golgi apparatus, etc.), cellular compartment (e.g., chromatin), microorganism (e.g., bacterium, virus, or fungus), virus, and vesicle (e.g., lysosome, peroxisome), small molecule, protein complex, protein aggregate, or a macromolecule). In embodiments, the cellular component is a biomolecule.

The term “morphology” or “morphological characteristic” of a cell as used herein generally refers to the form, structure, and/or configuration of the cell. The morphology of a cell can comprise one or more aspects of a cell's appearance, such as, for example, shape, size, arrangement, form, structure, pattern(s) of one or more internal and/or external parts of the cell, or shade (e.g., color, greyscale, etc.). Non-limiting examples of a shape of a cell can include, but are not limited to, circular, elliptic, shmoo-like, dumbbell, star-like, flat, scale-like, columnar, invaginated, having one or more concavely formed walls, having one or more convexly formed walls, prolongated, having appendices, having cilia, having angle(s), having corner(s), etc. A morphological feature of a cell may be visible with treatment of a cell (e.g., small molecule or antibody staining). Alternatively, the morphological feature of the cell may not and need not require any treatment to be visualized in an image or video.

The term “partition” as used herein generally refers to a space or volume that may be suitable to contain one or more species or conduct one or more reactions. A partition may be a physical compartment, such as a droplet (e.g., a droplet in an emulsion), a bead, a well, a container, a channel, etc. A partition may isolate space or volume from another space or volume. In some cases, a partition may be a single compartment partition. In some cases, a partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “emulsion” as used herein generally refers to a stable suspension of two incompatible fluid materials, where one fluid (e.g., an aqueous liquid, such as water or buffer) is suspended or dispersed as minute particles or globules in another fluid (e.g., a non-aqueous liquid, such as oil). The suspended fluid can be a carrier for, e.g., one or more cells of interest.

An emulsion can be, for example, oil-in-water (o/w), water-in-oil (w/o), water-in-oil-in-water (w/o/w), or oil-in-water-in-oil (o/w/o) dispersions or particles. A water-in-oil emulsion may be referred to as an aqueous droplet. Non-limiting examples of an emulsion can include various lipid structures, such as unilamellar, paucilamellar, and multilamellar lipid vesicles, micelles, and lamellar phases. An emulsion can be a microemulsion. An emulsion can be a nanoemulsion.

An emulsion can comprise a single droplet. An emulsion can comprise a plurality of droplets (e.g., at least about 2 droplets, at least about 5 droplets, at least about 10 droplets, at least about 15 droplets, at least about 20 droplets, at least about 30 droplets, at least about 40 droplets, at least about 50 droplets, at least about 60 droplets, at least about 70 droplets, at least about 80 droplets, at least about 90 droplets, at least about 100 droplets, or more).

### I. Systems, Methods, and Compositions for Cell Analysis

One or more morphological properties of a cell can be used to, for example, study cell type and cell state, or to diagnose diseases. In some cases, cell shape can be one of the markers of cell cycle. Eukaryotic cells can show physical changes in shape which can be cell-cycle dependent, such as a yeast cell undergoing budding or fission. In some cases, cell shape can be an indicator of cell state and, thus, can be an indicator used for clinical diagnostics. In some cases, shape of a blood cell may change due to many clinical conditions, diseases, and medications (e.g., changes in red blood cells'morphologies resulting from parasitic infections). Additional examples of the morphological properties of the cell that can be used to analyze the cell can include, but are not limited to, features of cell membrane, nuclear-to-cytoplasm ratio, nuclear envelope morphology, and chromatin structure Methods, systems, and databases provided herein can be used analyze cells (e.g., previously uncharacterized or unknown cells) based on (e.g., solely on) such morphological properties of the cells. In some aspects of the present disclosure, one or more cells can be stained with a tag prior to imaging the one or more cells (e.g., to capture one or more images of each cell), and determining the presence, absence, or pattern of such tag in the image(s) can enhance scalability and/or accuracy of analyzing the one or more cells.

In some aspects of the present disclosure, even in the presence of the tag in the cells, analysis of the images of the cells (e.g., morphology-based analysis as provided herein) may be independent of the tag. Rather, the tag can be used subsequently (e.g., subsequent to imaging and/or analysis of images/videos of the cell) to subject the cells to one or more additional analytic methods, such as omics.

In some aspects of the present disclosure, analyzing a cell based on one or more images of the cell and one or more morphological features of the cells extracted therefrom can be complemented (e.g., to enhance scalability and/or accuracy) by subjecting the cell to one or more additional analytic methods, such as omics. In some cases, subjecting cells (e.g., sorted cells from an initial population of cells) to two or more different analysis methods (e.g., imaging and one or more omics as disclosed herein) can uncover unique or new parameters to define a cell or a collection of cells (e.g., clusters of cells) that would otherwise not be identified in other methods. In some cases, the one or more additional analytic methods can analyze one or more analytes of the cells (e.g., subsequent to morphological analysis and sorting) such as, for example, cell-free DNA, cell-free RNA (e.g., miRNA or mRNA), proteins, carbohydrates, autoantibodies, and/or metabolites. In some cases, the one or more additional analytic methods can include whole-genome sequencing, whole-genome bisulfite sequencing or EM-seq, small-RNA sequencing, and/or quantitative immunoassay. For example, the one or more additional analytic methods can include gene sequencing method, such as an assay for transposase-accessible chromatin using sequencing (ATAC-seq) method, a micrococcal nuclease sequencing (MNase-seq) method, a deoxyribonuclease hypersensitive sites sequencing (DNase-seq) method, or a chromatin immunoprecipitation sequencing (ChIP-seq) method. Any sequencing data generated from the sequencing method (e.g., data that can be correlated back with cell morphological analysis) can be directed to or derived from exons, selected genes, genomic regions, variants, or a combination thereof. Such genomic regions can include one or more polymorphisms, sets of genes, sets of regulatory elements, micro-deletions, homopolymers, simple tandem repeats, regions of high GC content, regions of low GC content, paralogous regions, or a combination thereof. The one or more polymorphisms can include one or more insertions, deletions, structural variant junctions, variable length tandem repeats, single nucleotide variants (SNV), copy number variants (CNV), single nucleotide polymorphism (SNP), or a combination thereof.

In some embodiments, at least one cell can be imaged in a cell flow system. In some cases, the at least one cell can be stained (e.g., at least a portion of the cell, such as a cellular component as disclosed herein, can be stained) using at least one heterologous marker (e.g., a dye). In some cases, the at least one cell (e.g., that is stained) can be rotated in a field of view of an imaging device operatively coupled to the cell flow system. In some cases, the at least one cell can be imaged (e.g., via the imaging device) to create at least one cell image.

In some embodiments, the at least one cell can be stained and subsequently washed (e.g., using a buffer) to remove some of substantially all of any free heterologous marker that is not staining the least one cell. In some embodiments, the at least one cell may not and need not be washed upon being contacted by a medium comprising the heterologous marker.

In some embodiments, the at least one cell can be subjected to rotation by at least or up to about 0.1 microsecond (μs), at least or up to about 0.2 μs, at least or up to about 0.5 μs, at least or up to about 1 μs, at least or up to about 2 μs, at least or up to about 5 μs, at least or up to about 10 μs, at least or up to about 20 μs, at least or up to about 50 μs, at least or up to about 100 μs, at least or up to about 200 μs, at least or up to about 500 μs, at least or up to about 1 millisecond (ms), at least or up to about 2 ms, at least or up to about 5 ms, at least or up to about 10 ms, at least or up to about 20 ms, at least or up to about 50 ms, at least or up to about 100 ms, at least or up to about 200 ms, at least or up to about 500 ms, at least or up to about 1 second (sec), at least or up to about 2 seconds, at least or up to about 5 seconds, or at least or up to about 10 seconds after staining of the at least one cell by the at least one heterologous marker.

In some embodiments, the at least one cell can be subjected to imaging (e.g., via the imaging device) by at least or up to about 0.1 microsecond (μs), at least or up to about 0.2 μs, at least or up to about 0.5 μs, at least or up to about 1 μs, at least or up to about 2 μs, at least or up to about 5 μs, at least or up to about 10 μs, at least or up to about 20 μs, at least or up to about 50 μs, at least or up to about 100 μs, at least or up to about 200 μs, at least or up to about 500 μs, at least or up to about 1 millisecond (ms), at least or up to about 2 ms, at least or up to about 5 ms, at least or up to about 10 ms, at least or up to about 20 ms, at least or up to about 50 ms, at least or up to about 100 ms, at least or up to about 200 ms, at least or up to about 500 ms, at least or up to about 1 second (sec), at least or up to about 2 seconds, at least or up to about 5 seconds, or at least or up to about 10 seconds subsequent to the staining of the at least one cell by the at least one heterologous marker.

In some embodiments, the at least one cell image can comprise a single image of a cell. In some embodiments, the at least one cell image can comprise a plurality of images of a cell, e.g., at least or up to about 2 images, at least or up to about 3 images, at least or up to about 4 images, at least or up to about 5 images, at least or up to about 6 images, at least or up to about 7 images, at least or up to about 8 images, at least or up to about 9 images, at least or up to about 10 images, at least or up to about 15 images, at least or up to about 20 images, at least or up to about 30 images, at least or up to about 40 images, at least or up to about 50 images, or at least or up to about 100 images of the at least one cell. The plurality of images of the cell can be from the same angle or surface of the cell. Alternatively or in addition to, the plurality of images of the cell can be form different angles or surfaces of the cell (e.g., via capturing the plurality of images while rotating the cell).

In some embodiments, the at least one cell can be directed to flow in a flow cell of the cell flow system. The staining of the at least one cell, the rotating of the at least one cell, and the imaging of the at least one cell, as disclosed herein, can be performed in a single flow channel (e.g., a microfluidic channel). Alternatively, such staining, rotating, and imaging can be done in a plurality of flow channels. For example, the at least one cell can be stained in a first flow channel, and subsequently the at least one cell that is stained can be directed to flow to a second flow channel (that is in fluid communication with the first flow channel) for rotating and imaging of the at least one cell.

In some embodiments, the staining of the at least one cell by the at least one heterologous marker can enhance efficiency of imaging of the at least one cell. For example, the imaging device (e.g., a single sensor or a plurality of sensors, such as camera(s)) can detect the at least one heterologous marker (e.g., a dye) coupled to the at least one cell. Prior to, subsequently with, or subsequent to the detection of the at least one cell stained by the at least one heterologous marker, such detected cell can be subjected to label-free imaging via the imaging device. Non-limiting examples of the label-free imaging can include brightfield imaging and/or darkfield imaging. In some cases, a first imaging data can be generated based on the detection of the at least one heterologous marker coupled to the at least one cell, and a second imaging data can be generated based on the label-free imaging. The first imaging data and the second imaging data can be subsequently analyzed (e.g., compared) to generate a third imaging data, which can be usable for analyzing or partitioning (e.g., sorting) the at least one cell. Alternatively or in addition to, the first imaging data can be analyzed based at least in part on the second imaging data, or vice versa.

In some embodiments, a rate of imaging data processing to determine presence or absence of one or more heterologous marker coupled to a cell, as disclosed herein, can be at least or up to about 1,000 images per second (images/sec), at least or up to about 2,000 images/sec, at least or up to about 5,000 images/sec, at least or up to about 10,000 images/sec, at least or up to about 20,000 images/sec, at least or up to about 50,000 images/sec, at least or up to about 100,000 images/sec, at least or up to about 200,000 images/sec, at least or up to about 500,000 images/sec, at least or up to about 1,000,000 images/sec, at least or up to about 2,000,000 images/sec, at least or up to about 5,000,000 images/sec, or at least or up to about 10,000,000 images/sec.

In some embodiments, a rate of imaging data processing to analyze label-free imaging of a cell, as disclosed herein, can be aet least or up to about 1,000 images per second (images/sec), at least or up to about 2,000 images/sec, at least or up to about 5,000 images/sec, at least or up to about 10,000 images/sec, at least or up to about 20,000 images/sec, at least or up to about 50,000 images/sec, at least or up to about 100,000 images/sec, at least or up to about 200,000 images/sec, at least or up to about 500,000 images/sec, at least or up to about 1,000,000 images/sec, at least or up to about 2,000,000 images/sec, at least or up to about 5,000,000 images/sec, or at least or up to about 10,000,000 images/sec.

In some embodiments, a rate of corelating a partition to an imaging data (e.g. the image data itself or any cellular character or trait derived therefrom), for vice versa, as disclosed herein, can be aet least or up to about 1,000 partitions per second (partitions/sec), at least or up to about 2,000 partitions/sec, at least or up to about 5,000 partitions/sec, at least or up to about 10,000 partitions/sec, at least or up to about 20,000 partitions/sec, at least or up to about 50,000 partitions/sec, at least or up to about 100,000 partitions/sec, at least or up to about 200,000 partitions/sec, at least or up to about 500,000 partitions/sec, at least or up to about 1,000,000 partitions/sec, at least or up to about 2,000,000 partitions/sec, at least or up to about 5,000,000 partitions/sec, or at least or up to about 10,000,000 partitions/sec.

In some embodiments, a population of cells can be analyzed based at least in part on any cell image or cell imaging data as disclosed herein, and the population of cells can be sorted in silico, e.g., via plotting into a cell clustering map, e.g., a cell morphology map. Each cell cluster of the cell clustering map can be characterized to share common image class (e.g., as defined by exhibiting a common image characteristic, a common target feature, and/or a common cellular component). For example, the cell clustering map can be based on the first imaging data (e.g., from detection of the at least one heterologous marker coupled to the at least one cell), the second imaging data (e.g., from the label-free imaging of the at least one cell), or both.

In some embodiments, the staining of the at least one cell by the at least one heterologous marker can enhance analysis of the at least one cell image, e.g., to identify one or more characteristics of the at least one cell (e.g., a morphological characteristic), and/or to classify the at least one cell. In some cases, analyzing the at least one cell image that is indicative of presence or absence of the at least one heterologous marker can enhance quality (e.g., resolution) of the at least one cell image, thereby enhancing analysis of the at least one cell, e.g., to classify the at least one cell. For example, staining the membrane of the at least one cell with a dye (e.g., Carbocyanine dyes) or staining for viability of the at least one cell (e.g., calcein-AM for live and ethidium homodimer-1 for dead) can enhance resolution or visibility of the at least one cell in the at least one cell image, thereby enhancing cell morphology analysis of the at least one cell image.

In some embodiments, the at least one cell can be delivered to a partition, and the partition can be a part (e.g., a channel) of the cell flow system. For example, subsequent to the imaging, the at least one cell can be directed to the partition. e.g., via a cell sorter in fluid communication with the cell imaging flow channel and the partition. The partition can comprise a reservoir (e.g., a collection container). The cell flow system can comprise a single partition.

The cell flow system can comprise a plurality of partitions (e.g., at least 1, at least 2, at least 3, at least 4, at least 5, at least 6, at least 7, at least 8, at least 9, at least 10, or more partitions). In some cases, the at least one cell can be directed to a partition without analysis of the at least one cell (e.g., via the order that the at least one cell has arrived to the imaging flow channel or the sorter). In some cases, the at least one cell can be directed to a partition based on one or more properties or characteristics of the at least one cell. The one or more properties/characteristics can be based on the at least one cell image (e.g., obtained via the imaging device subsequent to staining the cell using the at least one heterologous marker). Alternatively or in addition to, the one or more properties/characteristics can be based on data that is different from the at least one cell image, e.g., one or more properties of the at least one cell obtained by a sensor different from the imaging device. Such data can be independent of the at least one heterologous marker utilized to stain the at least one cell (e.g., size of the cell, shape of the cell, etc.).

In some embodiments, a cell as disclosed herein (e.g., stained or not stained by a heterologous marker) can be directed to (or delivered to) a partition as disclosed herein, by at least or up to about 0.1 microsecond (μs), at least or up to about 0.2 μs, at least or up to about 0.5 μs, at least or up to about 1 μs, at least or up to about 2 μs, at least or up to about 5 μs, at least or up to about 10 μs, at least or up to about 20 μs, at least or up to about 50 μs, at least or up to about 100 μs, at least or up to about 200 μs, at least or up to about 500 μs, at least or up to about 1 millisecond (ms), at least or up to about 2 ms, at least or up to about 5 ms, at least or up to about 10 ms, at least or up to about 20 ms, at least or up to about 50 ms, at least or up to about 100 ms, at least or up to about 200 ms, at least or up to about 500 ms, at least or up to about 1 second (sec), at least or up to about 2 seconds, at least or up to about 5 seconds, or at least or up to about 10 seconds subsequent to imaging of the cell.

In some embodiments, a cell that is stained by a heterologous marker and is subsequently imaged, as disclosed herein, can be directed to (or delivered to) a partition as disclosed herein, by at least or up to about 0.1 microsecond (μs), at least or up to about 0.2 μs, at least or up to about 0.5 μs, at least or up to about 1 μs, at least or up to about 2 μs, at least or up to about 5 μs, at least or up to about 10 μs, at least or up to about 20 μs, at least or up to about 50 μs, at least or up to about 100 μs, at least or up to about 200 μs, at least or up to about 500 μs, at least or up to about 1 millisecond (ms), at least or up to about 2 ms, at least or up to about 5 ms, at least or up to about 10 ms, at least or up to about 20 ms, at least or up to about 50 ms, at least or up to about 100 ms, at least or up to about 200 ms, at least or up to about 500 ms, at least or up to about 1 second (sec), at least or up to about 2 seconds, at least or up to about 5 seconds, or at least or up to about 10 seconds subsequent to staining of the cell by the heterologous marker.

In some embodiments, a cell can be lysed prior to, simultaneously with, or subsequent to delivery of the cell (or one or more cellular components of the cell) to the partition. For example, the cell can be contacted with a cell lysis agent (e.g., a cell lysis buffer such as sodium dihydrogen phosphate/disodium hydrogen phosphate buffer, Tris-HCl buffer, HEPES-NaOH buffer, etc.) prior to, simultaneously with, or subsequent to delivery of the cell to the partition.

In another example, the cell can be stimulated (e.g., sonicated) for cell lysis prior to, simultaneously with, or subsequent to delivery of the cell to the partition.

In some embodiments, the partition as disclosed herein can comprise a closed end, e.g., not in fluid communication with any additional flow channel other than the source of the at least one cell that is delivered into the partition. For example, the partition can be a well. Alternatively, the partition can comprise one or more additional openings that are in fluid communication with an additional flow channel. For example, collected cells in the partition can be directed to flow via the additional flow channel into another analysis module to analyze one or more components (or analytes) derived from the cell, such as an omics module (e.g., genomics, transcriptomics, or proteomics, etc.).

In some embodiments, the at least one cell that is delivered to the partition can be in a droplet. The droplet can be formed prior to, concurrently with, or subsequent to the delivery of the at least one cell into the partition. The droplet can be formed prior to, currently with, or subsequent to the staining of the at least one cell with the heterologous marker. For example, the droplet can be formed prior to imaging of the at least one cell via the imaging device. A droplet as disclosed herein can have, on average, a single cell (e.g., a single cell droplet). Alternatively, a droplet can comprise, on average, a plurality of cells, e.g., at least two, at least three, at least four, at least five, or more cells per droplet. Yet in another alternative, a droplet can comprise less than the entirety of the at least one cell, e.g., the droplet can be formed after breaking down (or lysing) the least one cell into different parts (or components), such that the droplet comprises some of the nucleic acid molecules (e.g., that are tagged with the heterologous marker) derived from the at least one cell.

In an example, (i) a cell (e.g., an imaged cell) or one or more cellular components thereof and (ii) a heterologous marker (e.g., a tag, such as a barcode) can be colocalized into a droplet, as disclosed herein, and the droplet can be directed (e.g., sorted) into the partition.

In some cases, a droplet as disclosed herein can be stained with (or tagged by) a heterologous marker, such that each individual droplet can be identified and/or tracked subsequent to formation of each droplet. One or more cells (or cellular fragments thereof) encapsulated within the droplet may or may not be stained by an additional heterologous marker.

In some embodiments, a droplet as disclosed herein can be in an emulsion (e.g., a single cell emulsion). The emulsion can be a microemulsion, e.g., having a mean particle size greater than or equal to about 5 micrometers (μm). The emulsion can be in a nanoemulsion, e.g., having a mean particle size less than about 5 μm. The emulsion can be characterized by having a mean particle size of at least or up to about 1 nanometer (nm), at least or up to about 2 nm, at least or up to about 5 nm, at least or up to about 10 nm, at least or up to about 20 nm, at least or up to about 30 nm, at least or up to about 40 nm, at least or up to about 50 nm, at least or up to about 60 nm, at least or up to about 70 nm, at least or up to about 80 nm, at least or up to about 90 nm, at least or up to about 100 nm, at least or up to about 200 nm, at least or up to about 300 nm, at least or up to about 400 nm, at least or up to about 500 nm, at least or up to about 600 nm, at least or up to about 700 nm, at least or up to about 800 nm, at least or up to about 900 nm, at least or up to about 1,000 nm, at least or up to about 2,000 nm, or at least or up to about 5,000 nm.

In some embodiments, prior to, during, or subsequent to the delivery of the at least one cell to the partition, the partition can be correlated to the at least one cell image of the at least one cell. For example, the partition can be digitally assigned to digital data derived from the at least one cell image (e.g., the at least one cell image in its entirety, one or more characteristics derived from the at least one cell image, such as a morphological characteristic, the heterologous marker staining the at least one cell, etc.). As such, a cell or a collection of cells of interest can be retrieved by selecting a partition with a correlated digital data of interest.

In some embodiments, a first cell population can be contacted (e.g., stained) by a heterologous marker (e.g., a dye) that distinguishes a target feature (e.g., a dye target feature) of a subset of the first cell population. The first cell population can be imaged to generate imaging data (e.g., based on one or more images of the first cell population), and an image feature (or image characteristic) of the first population that correlates to binding of the heterologous marker (e.g., binding of the dye) can be identified based on the imaging data. Subsequently, a second cell population can be imaged, and the second cell population can be analyzed and/or sorted based on presence or absence of the image feature. In some cases, the second cell population can be contacted by the same heterologous marker prior to imaging of the second cell population. In some cases, the second cell population may not and need not be contacted by the same heterologous marker prior to the imaging (e.g., the second cell population may be stained with a different heterologous marker for different label-based imaging, or may not be stained with any marker for label-free imaging). Accordingly, the first cell population can be utilized to train an algorithm (e.g., a machine learning algorithm or an artificial intelligence algorithm, such as a classifier) to analyze or sort a subsequent cell population based at least in part on presence or absence of the heterologous marker in the subsequent cell population.

In some embodiments, a size of the first cell population (or a number of cells in the first cell population) can be sufficient to train such algorithm. The size of the first cell population can be at least or up to about 1 cell, at least or up to about 2 cells, at least or up to about 5 cells, at least or up to about 10 cells, at least or up to about 15 cells, at least or up to about 20 cells, at least or up to about 30 cells, at least or up to about 40 cells, at least or up to about 50 cells, at least or up to about 60 cells, at least or up to about 70 cells, at least or up to about 80 cells, at least or up to about 90 cells, at least or up to about 100 cells, at least or up to about 200 cells, at least or up to about 300 cells, at least or up to about 400 cells, at least or up to about 500 cells, at least or up to about 600 cells, at least or up to about 700 cells, at least or up to about 800 cells, at least or up to about 900 cells, or at least or up to about 1,000 cells.

In some embodiments, the target feature and the image feature can be correlated to each another, similar to each another, or substantially the same as each other. In some embodiments, the image feature can comprise at least or up to about 1 image feature, at least or up to about 2 image features, at least or up to about 3 image features, at least or up to about 4 image features, at least or up to about 5 image features, at least or up to about 6 image features, at least or up to about 7 image features, at least or up to about 8 image features, at least or up to about 9 image features, at least or up to about 10 image features, at least or up to about 15 image features, or at least or up to about 20 image features. An image feature can be (i) various morphological features of a cell, or (ii) presence or absence of one or more cellular components. Non-limiting examples of an image feature can include a cell surface protein, cell size, cell shape, nucleus size, nucleus shape, surface topology, cytoplasmic feature, nucleolus, cytoplasmic organelle, etc.

In some embodiments, the heterologous marker can selectively bind to a subset of the first population of cells. The subset can be at least or up to about 1%, at least or up to about 2%, at least or up to about 5%, at least or up to about 10%, at least or up to about 15%, at least or up to about 20%, at least or up to about 30%, at least or up to about 40%, at least or up to about 50%, at least or up to about 60%, at least or up to about 70%, at least or up to about 80%, at least or up to about 85%, at least or up to about 90%, at least or up to about 95%, or at least or up to about 99% of the first population of cells.

In some embodiments, the first cell population and the second cell population can be derived from a common source (or the same source), such as, for example, a common biological sample derived from a subject. Alternatively, the first cell population and the second cell population can be derived from distinct (or different) sources. The distinct sources can be, for example, the same biological sample type (e.g., a blood sample) derived from a single subject at different time points, different biological samples derived from a single subject (e.g., a blood sample and a biopsy from a solid tissue), the same biological sample type (e.g., a blood sample) derived from different subjects, etc.

In some embodiments, sorting the second population can comprise successively imaging a linear file of cells (or a linear streamline of cells) of the second population, and differentially depositing cells (e.g., differentially sorting such cells into one or more partitions as disclosed herein) of the linear file of cells, based at least in part on presence or absence of the image feature. For example, based on the presence or absence of the image feature in the cells of the linear file of cells, each of the linear file of cells can be directed into different partitions (e.g., different reservoirs). As described herein, each partition can be corrected with a cell imaging data or an analysis thereof (e.g., image feature derived from the cell imaging data) of the cell that is collected in the partition.

In some embodiments, a population of cells can be enriched for imaged cells that share a common image characteristic (or image feature), as disclosed herein. In some cases, a cell can be imaged to obtain a cell image, and the cell image can be compared to a database. Based at least in part on comparison of the cell image to the database, the cell can be delivered to a partition, as disclosed herein. For examples, the partition can comprise a plurality of cells, wherein at least some of the plurality of cells can have an image characteristic (e.g., the common image characteristic or a different image characteristic) that exhibits a degree of correlation (e.g., a correlation factor) to an independent image of the database. In addition, the partition can be correlated to the cell image prior to, simultaneously with, or subsequent to delivering the cell to the partition. Accordingly, the cell can be sorted along with the plurality of cells for bulk sorting.

In some embodiments, the cell or the plurality of cells can be stained with one or more heterologous markers (e.g., a dye, a barcode, etc.) as disclosed herein, e.g., to enhance imaging and/or analysis of the cell or the plurality of cells. In some embodiments, imaging the cell for obtaining the cell image can be label-based imaging (e.g., detecting presence or absence of the heterologous marker) or label-free imaging (e.g., brightfield imaging), as disclosed herein.

In some embodiments, the database can comprise a single independent image or a plurality of independent images. The plurality of images can comprise at least or up to about 2 images, at least or up to about 3 images, at least or up to about 4 images, at least or up to about 5 images, at least or up to about 6 images, at least or up to about 7 images, at least or up to about 8 images, at least or up to about 9 images, at least or up to about 10 images, at least or up to about 15 images, at least or up to about 20 images, at least or up to about 30 images, at least or up to about 40 images, at least or up to about 50 images, at least or up to about 60 images, at least or up to about 70 images, at least or up to about 80 images, at least or up to about 90 images, at least or up to about 100 images, at least or up to about 150 images, at least or up to about 200 images, at least or up to about 300 images, at least or up to about 400 images, at least or up to about 500 images, at least or up to about 600 images, at least or up to about 700 images, at least or up to about 800 images, at least or up to about 900 images, at least or up to about 1,000 images, at least or up to about 2,000 images, at least or up to about 5,000 images, at least or up to about 10,000 images, at least or up to about 20,000 images, or at least or up to about 50,000 images. The plurality of independent images can be obtained from a single cell or from a plurality of cells. An independent image can be selected based on one or more image characteristics that can be or that are retrievable from the independent image. For example, an image characteristic can be indicative of presence or absence of (i) a cellular component and/or (ii) a cellular morphology.

In some embodiments, the common image characteristic can comprise at least or up to about 1 image characteristic, at least or up to about 2 image characteristics, at least or up to about 3 image characteristics, at least or up to about 4 image characteristics, at least or up to about 5 image characteristics, at least or up to about 6 image characteristics, at least or up to about 7 image characteristics, at least or up to about 8 image characteristics, at least or up to about 9 image characteristics, or at least or up to about 10 image characteristics. In some case, a single image characteristic can be obtained from the cell image. In some cases, a plurality of different image characteristics can be obtained from the cell image.

In some embodiments, the cell and/or at least some of the plurality of cells can be characterized (e.g., via machine learning or artificial intelligence algorithms) to have an image characteristic, and such image characteristic can exhibit a degree of correlation to the independent image of the database. The degree of correlation (e.g., a degree of similarity) between such image characteristic and the independent image can be at least or up to about 1%, at least or up to about 2%, at least or up to about 5%, at least or up to about 10%, at least or up to about 20%, at least or up to about 30%, at least or up to about 40%, at least or up to about 50%, at least or up to about 60%, at least or up to about 70%, at least or up to about 80%, at least or up to about 85%, at least or up to about 90%, at least or up to about 95%, at least or up to about 99%, or about 100%.

In some embodiments, a partition as disclosed herein can comprise a plurality of cells (e.g., a plurality of sorted cells). The plurality of cells can be disposed within the partition in an ordered fashion (e.g., organized into one or more streamlines in a sequential manner).

Alternatively, the plurality of cells may not and need not be disposed in any ordered fashion. As described herein, the plurality of cells in the partition can be in an emulsion, either individually (e.g., single cell emulsion) or collectively (e.g., multi-cell emulsion).

In some embodiments, the amount (or proportion) of cells in the partition that do not have an image characteristic similar to the independent image of the database (e.g., the common image characteristic) may be at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 9%, at most about 8%, at most about 7%, at most about 6%, at most about 5%, at most about 4%, at most about 3%, at most about 2%, or at most about 1%. In some embodiments, the amount (or proportion) of cells in the partition that have an image characteristic similar to the independent image of the database (e.g., the common image characteristic) may be at least about 1%, at least about 5%, at least about 10%, at least about 15%, at least about 20%, 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or substantially 100%.

In some embodiments, a first image of a first cell of a population of cells as disclosed herein can be generated (e.g., via an imaging device), and the first cell can be delivered to a first partition. A second image of a second cell of the population of cells can be imaged (e.g., subsequent to generation of the first image and/or delivery of the first cell to the first partition). When the second image is similar to the first image (e.g., having a correlation as disclosed herein), the second cell can be delivered to the first partition having the first cell. Accordingly, the population of cells can be sorted in bulk. In some cases, the first image and/or the second image can be stored in a database operatively coupled to at least the first partition. The first image and/or the second image can be used as an independent image for sorting any subsequent cell or population of cells, as disclosed herein.

In some embodiments, the first image can be characterized (e.g., via machine learning or artificial intelligence algorithms) to have a first image characteristic and the second image can be characterized to have a second image characteristic that exhibits a degree of correlation to the first image characteristic. The degree of correlation (e.g., a degree of similarity) between the first and second image characteristics can be at least or up to about 1%, at least or up to about 2%, at least or up to about 5%, at least or up to about 10%, at least or up to about 20%, at least or up to about 30%, at least or up to about 40%, at least or up to about 50%, at least or up to about 60%, at least or up to about 70%, at least or up to about 80%, at least or up to about 85%, at least or up to about 90%, at least or up to about 95%, at least or up to about 99%, or about 100%.

In some embodiments, a third image of a third cell of the population of cells can be imaged (e.g., subsequent to generation of the first image and/or delivery of the first cell to the first partition). When the third image is dissimilar from the first image of the first cell, the third cell can be delivered to a second partition that is different from the first partition. In addition, the second partition can be correlated to the third image, or vice versa, as disclosed herein.

In some embodiments, a degree of similarity between a third image characteristic of the third cell and the first image characteristic of the first cell (or an average image characteristic of at least the first cell and the second cell) can be less than about 70%, less than about 60%, less than about 50%, less than about 45%, less than about 40%, less than about 35%, less than about 30%, less than about 25%, less than about 20%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, or less than about 1%, e.g., based on one or more machine learning or artificial intelligence algorithms as disclosed herein.

In some embodiments, a partition as disclosed herein can comprise a plurality of cells, and a majority of the plurality of cells can share a common image characteristic, a common target feature, and/or a common cellular component. The majority can be at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the plurality of cells in the partition.

In some embodiments, a partition as disclosed herein can comprise a processing agent, such that a cell that is directed to (e.g., sorted to) the partition can be contacted by the processing agent. Non-limiting examples of the processing agent can include a heterologous marker, a cell lysis reagent, a cell fixative, a reverse transcriptase, an endonuclease (e.g., a nucleic acid guided endonuclease, such as a CRISPR/Cas protein), a pharmaceutical (e.g., a drug to elicit a desired effect in the cell), a cell division inhibitor, a cell growth inhibitor, and/or a cell differentiation inhibitor. For example, the cell can be fixed by the cell fixative to preserve integrity of the cell (e.g., structural integrity) in the partition. In another example, the cell can be contacted by the cell division inhibitor, the cell growth inhibitor, and/or the cell differentiation inhibitor, such that the state of the cell (e.g., cell cycle, degree of stemness of the cell, cell differentiation type, etc.) can be preserved in the partition.

In some embodiments, a plurality of cells characterized to exhibit a common image characteristic, a common target feature, and/or a common cellular component can be directed to a common partition. In some embodiments, a plurality of cells characterized to exhibit different image characteristics, different target features, and/or different cellular components can be directed to a common partition. In such case, for example, the plurality of cells (or cellular components thereof) can be tagged with a heterologous marker (e.g., a barcode), to allow identification or tracking of each of the plurality of cells (or the cellular components thereof) in the partition, or during any subsequent analysis.

In some embodiments, a reservoir as disclosed herein can comprise a plurality of cells, and one or more of the plurality of cells (e.g., substantially all of the plurality of cells) in the partition can be individually imaged cells (e.g., to generate imaging data prior to or during sorting into the partition). Each individually imaged cell may or may not be labeled with the heterologous marker, as disclosed herein.

In some embodiments, a heterologous marker as disclosed herein can exhibit specific binding to a target polynucleotide sequence derived from a cell (e.g., an individually imaged cell). In some cases, the heterologous marker can comprise a polynucleotide sequence that exhibits at least about 50%, at least about 55%, at least about 60%, at least about 65%, at least about 70%, at least about 75%, at least about 80%, at least about 85%, at least about 90%, at least about 95%, or at least about 99% sequence identity (or sequence complementarity) to the target polynucleotide sequence. In some cases, the heterologous marker can further comprise a tag (e.g., a barcode) coupled to the polynucleotide sequence of the heterologous marker.

In some embodiments, a heterologous marker can be added to a partition as disclosed herein, such as that at least a portion of a cell (e.g., one or more cellular components of the cell) disposed or sorted into the partition can be stained by the heterologous marker. For example, the heterologous marker can be a barcode that can be coupled to a target polypeptide (e.g., a protein or a fragment thereof) or a target polynucleotide (e.g., a target genomic DNA fragment) derived from the cell. As described herein, the heterologous marker can be used to further analyze the cell or the one or more cellular components of the cell (e.g., via omics analysis).

In some embodiments, an image of a cell (e.g., an imaged cell) as disclosed herein can be digitally labeled, such that the image can be correlated (e.g., tracked) with the individually imaged cell or one or more cellular components thereof that are (i) labeled with a heterologous marker, (ii) encapsulated in an emulsion that is labeled with a heterologous marker, and/or (iii) directed or sorted into a partition that is digitally labeled with an image characteristic of interest.

In some embodiments, a partition as disclosed herein can comprise a plurality of cells (e.g., a plurality of individually imaged cells that may or may not be labeled with a heterologous marker). The plurality of cells can comprise at least or up to about 1 cell, at least or up to about 2 cells, at least or up to about 5 cells, at least or up to about 10 cells, at least or up to about 15 cells, at least or up to about 20 cells, at least or up to about 30 cells, at least or up to about 40 cells, at least or up to about 50 cells, at least or up to about 60 cells, at least or up to about 70 cells, at least or up to about 80 cells, at least or up to about 90 cells, at least or up to about 100 cells, at least or up to about 200 cells, at least or up to about 300 cells, at least or up to about 400 cells, at least or up to about 500 cells, at least or up to about 600 cells, at least or up to about 700 cells, at least or up to about 800 cells, at least or up to about 900 cells, at least or up to about 1,000 cells, at least or up to about 2,000 cells, at least or up to about 5,000 cells, at least or up to about 10,000 cells, at least or up to about 20,000 cells, at least or up to about 50,000 cells, at least or up to about 100,000 cells, at least or up to about 200,000 cells, at least or up to about 500,000 cells, or at least or up to about 1,000,000 cells.

In some embodiments, a partition can comprise a plurality of cells (e.g., a plurality of individually imaged cells). At least a portion of the plurality of cells can be grouped into a common image class (e.g., as defined by exhibiting a common image characteristic, a common target feature, and/or a common cellular component). The at least the portion of the plurality of cells can be at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, or at least or up to about 99%.

In some embodiments, the at least one cell can be delivered to one or more partitions. The one or more partitions can be disposed in an emulsion. For example, the one or more partitions can be one or more aqueous droplets (e.g., a plurality of aqueous droplets) dispersed in an oil solvent carrier. A partition of the one or more partitions can comprise one or more cells (e.g., one or more individually imaged cells that are subjected to imaging prior to being disposed into the partition) as disclosed herein, e.g., at least or up to about 1 cell, at least or up to about 2 cells, at least or up to about 3 cells, at least or up to about 4 cells, at least or up to about 5 cells, at least or up to about 6 cells, at least or up to about 7 cells, at least or up to about 8 cells, at least or up to about 9 cells, at least or up to about 10 cells, at least or up to about 15 cells, at least or up to about 20 cells, at least or up to about 25 cells, at least or up to about 30 cells, at least or up to about 40 cells, or at least or up to about 50 cells.

In some embodiments, a majority of the one or more cells (e.g., the one or more individually imaged cells) can be mapped to a cluster within a cell clustering map, as disclosed herein. Alternatively, a majority of the one or more cells (e.g., the one or more individually imaged cells) may not be capable of being mapped to a cluster within a cell clustering map. The majority can be at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the one or more cells.

In some embodiments, a heterologous marker can be coupled to (e.g., directly coupled to) a cell or one or more cellular components of the cell. Alternatively, a heterologous marker can be coupled to a partition (e.g., a surface of a droplet in an emulsion), or disposed within the partition along with the cell. For example, a cell (e.g., an individually imaged cell) disposed in a first solvent, and a heterologous marker can be dispersed in a second solvent, wherein the first and second solvents are miscible. The first solvent comprising the cell and the second solvent comprising the heterologous marker can be mixed, then be contacted with an additional solvent that is not miscible with the first or second solvent, to form an emulsion comprising a droplet that comprises the cell and the heterologous marker.

In some embodiments, a population of cells can be partitioned into a plurality of partitions (e.g., wells, droplets, beats, etc.), such that each partition comprises a plurality of cells. A plurality of cells can be co-partitioned into a single partition. In some cases, each cell (or a cellular component of the cell) can be tagged with a heterologous marker (e.g., a barcode). The population of cells can be partitioned, such that between cells in a given partition, the heterologous markers coupled thereto may be the same, but between different partitions, the cells can have different heterologous markers.

In some embodiments, a heterologous marker can comprise a barcode, e.g., a nucleic acid barcode. Nucleic acid barcode sequences as disclosed herein can comprise from about 6 to about 20 or more nucleotides within each nucleic acid barcode sequence (e.g., oligonucleotides or barcode oligo). The nucleic acid barcode sequences can include from about 6 to about 20, about 30, about 40, about 50, about 60, about 70, about 80, about 90, about 100, or more nucleotides. In some cases, the length of a barcode sequence may be about 6, about 7, about 8, about 9, about 10, about 11, about 12, about 13, about 14, about 15, about 16, about 17, about 18, about 19, about 20 nucleotides or longer. In some cases, the length of a barcode sequence may be at least about 6, about 7, about 8, about 9, about 10, about 11, about 12, about 13, about 14, about 15, about 16, about 17, about 18, about 19, about 20 nucleotides or longer. In some cases, the length of a barcode sequence may be at most about 6, about 7, about 8, about 9, about 10, about 11, about 12, about 13, about 14, about 15, about 16, about 17, about 18, about 19, about 20 nucleotides or shorter. These nucleotides may be completely contiguous, e.g., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, about 5, about 6, about 7, about 8, about 9, about 10, about 11, about 12, about 13, about 14, about 15, about 16 nucleotides or longer. In some cases, the barcode subsequence may be at least about 4, about 5, about 6, about 7, about 8, about 9, about 10, about 11, about 12, about 13, about 14, about 15, about 16 nucleotides or longer. In some cases, the barcode subsequence may be at most about 4, about 5, about 6, about 7, about 8, about 9, about 10, about 11, about 12, about 13, about 14, about 15, about 16 nucleotides or shorter.

In some embodiments, a nucleic acid barcode sequence can comprise one or more additional sequences useful in the processing of the cells or fragments thereof (e.g., from within the co-partitioning or across different partitions). The one or more additional sequences can include, e.g., targeted or random/universal amplification primer sequences for amplifying the genomic DNA, sequencing primers or primer recognition sites, hybridization or probing sequences, e.g., for identification of presence of the sequences or for pulling down barcoded nucleic acids, etc.

In some embodiments, a diverse barcode sequence library can be utilized, and the diverse barcode sequence library can comprise at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more.

In some embodiments, a cell or one or more fragments thereof that is partitioned, as disclosed herein, can be releasable from the partition, e.g., for an additional analysis thereof (e.g., sequencing or additional imaging).

In some embodiments, a partition as disclosed herein can be a part of a surface of a substrate (e.g., a plate). The surface can comprise a spot (e.g., a marked spot) to which a cell (e.g., an individually imaged cell) or fragments thereof can be spotted. The cell can be dispersed in a solvent (e.g., a aqueous solvent) prior to being directed to (e.g., spotted) to the spot. The cell can be disposed within an emulsion prior to being directed to the spot. In some cases, the cell can be tagged with a heterologous marker (e.g., barcode) prior to the spotting. In some cases, the spot can comprise one or more heterologous markers, and the cell or fragments of the cell can be tagged with the one or more heterologous markers upon spotting onto the surface. The surface can comprise at least 1 spot, at least 2 spots, at least 5 spots, at least 10 spots, at least 15 spots, at least 20 spots, at least 30 spots, at least 40 spots, at least 50 spots, at least 60 spots, at least 70 spots, at least 80 spots, at least 90 spots, at least 100 spots, at least 150 spots, at least 200 spots, at least 300 spots, at least 400 spots, at least 500 spots, at least 600 spots, at least 700 spots, at least 800 spots, at least 900 spots, at least 1,000 pots, or more. Each spot can comprise at least or up to about 1 heterologous marker, at least or up to about 2 heterologous markers, at least or up to about 3 heterologous markers, at least or up to about 4 heterologous markers, at least or up to about 5 heterologous markers, at least or up to about 6 heterologous markers, at least or up to about 7 heterologous markers, at least or up to about 8 heterologous markers, at least or up to about 9 heterologous markers, at least or up to about 10 heterologous markers, at least or up to about 15 heterologous markers, at least or up to about 20 heterologous markers, at least or up to about 30 heterologous markers, at least or up to about 40 heterologous markers, at least or up to about 50 heterologous markers, at least or up to about 60 heterologous markers, at least or up to about 70 heterologous markers, at least or up to about 80 heterologous markers, at least or up to about 90 heterologous markers, or at least or up to about 100 heterologous markers.

In some embodiments, cells (e.g., individually imaged cells) can be delivered or directed to one or more spots as disclosed herein, to print the cells or fragments thereof onto the one or more spots. In some cases, the one or more heterologous markers (e.g., barcodes) in a spot can be recorded, to associate the individually imaged cell (and/or one or more images from imaging of the individually imaged cell, imaging data derived from the one or more images, etc.) to the one or more heterologous markers in the spot. The recording can be performed prior to, simultaneously with, or subsequent to printing of the cell or fragments thereof onto the spot.

In some embodiments, each spot (e.g., comprising one or more heterologous markers, such as barcodes) can be printed with one or more cells, e.g., at least or up to about 1 individually imaged cell, at least or up to about 2 individually imaged cells, at least or up to about 3 individually imaged cells, at least or up to about 4 individually imaged cells, at least or up to about 5 individually imaged cells, at least or up to about 6 individually imaged cells, at least or up to about 7 individually imaged cells, at least or up to about 8 individually imaged cells, at least or up to about 9 individually imaged cells, at least or up to about 10 individually imaged cells, at least or up to about 11 individually imaged cells, at least or up to about 12 individually imaged cells, at least or up to about 13 individually imaged cells, at least or up to about 14 individually imaged cells, at least or up to about 15 individually imaged cells, at least or up to about 20 individually imaged cells, at least or up to about 25 individually imaged cells, at least or up to about 30 individually imaged cells, at least or up to about 40 individually imaged cells, at least or up to about 50 individually imaged cells, at least or up to about 100 individually imaged cells, at least or up to about 200 individually imaged cells, at least or up to about 500 individually imaged cells, at least or up to about 1,000 individually imaged cells, at least or up to about 2,000 individually imaged cells, at least or up to about 5,000 individually imaged cells, at least or up to about 10,000 individually imaged cells, at least or up to about 20,000 individually imaged cells, at least or up to about 50,000 individually imaged cells, at least or up to about 100,000 individually imaged cells, at least or up to about 200,000 individually imaged cells, or at least or up to about 500,000 individually imaged cells. When a plurality of cells is printed onto a common spot, the plurality of cells may be characterized (e.g., prior to the printing) to exhibit a common image class (e.g., as defined by exhibiting a common image characteristic, a common target feature, and/or a common cellular component).

In some embodiments, one or more subpopulations of a cell population can be assigned with one or more characteristics (e.g., indicative of presence of absence of one or more cellular components, one or more morphological characteristics, etc.). In some cases, a characteristic (e.g., a phenotypic characteristic, an image characteristic, etc.) of at least some cells of the cell population can be measured (e.g., via imaging, such as label-mediated imaging or label-free imaging, as disclosed herein), and the at least some cells of the cell population can be assigned to one subpopulation of at least two subpopulations. The one subpopulation of the at least two subpopulations can be correlated to (i) an independently expected subpopulation and/or (ii) a cell population dataset comprising dataset subpopulations having one or more known traits (or characteristics). Accordingly, at least a portion of the cell population (e.g., the one subpopulation) can be analyzed via bioinformatic mapping.

In some embodiments, measuring the characteristic of the at least some cells of the cell population can comprise imaging (e.g., via an imaging device as disclosed herein) the at least some cells to generate imaging data, wherein the imaging data can be analyzed to generate (e.g., extract) one or more image characteristics. The one or more image characteristics can be indicative of one or more phenotypic characteristic, e.g., a morphological characteristic as disclosed herein. Such characteristic(s) of the cells can be further analyzed (e.g., via machine learning or artificial intelligence algorithms) to assign the at least some cells to the one subpopulation of the at least two subpopulations. Such assigning can be substantially free of human input (e.g., automated). In some cases, prior to imaging, the at least some cells can be contacted by a heterologous marker (e.g., a dye). For example, the at least some cells can be contacted with at least one dye (e.g., at least 1, at least 2, at least 3, at least 4, at least 5, or more different dyes) to stain one or more cellular components of the cell. Presence or absence of the at least one dye in imaging data can be utilized to help selection of one or more cells from the at least some cells or analysis (e.g., classification) of the at least some cells.

In some embodiments, the at least two subpopulations can comprise at least or up to about 2, at least or up to about 3, at least or up to about 4, at least or up to about 5, at least or up to about 6, at least or up to about 7, at least or up to about 8, at least or up to about 9, at least or up to about 10, at least or up to about 15, or at least or up to about 20 subpopulations.

In some embodiments, assigning the at least some cells of the cell population to the one subpopulation of the at least two subpopulations can comprise directing one or more cells of the at least some cells of the cell population to a partition (e.g., a well, a droplet, a bead, a spot comprising a barcode, etc.) as disclosed herein.

In some embodiments, the correlating the one subpopulation to the independently expected subpopulation can comprise attributing at least one trait of the independently expected subpopulation to the at least some cells of the cell population. In some cases, one or more cells of the one subpopulation can be further analyzed (e.g., via omics as disclosed herein) to confirm or verify the attributed at least one trait. In some cases, the one subpopulation can be correlated with exhibiting (or expected to exhibit, e.g., via machine learning or artificial intelligence algorithms) the at least one trait of the independently expected subpopulation, while another subpopulation of the at least two subpopulations can be correlated with substantially lacking (or expected to lack, e.g., via machine learning or artificial intelligence algorithms) the at least one trait.

In some embodiments, the correlating the one population to the cell population dataset comprising the dataset subpopulations having the one or more known traits can comprise confirming or matching the trait(s) of cells of the one population and the trait(s) of cells of the dataset subpopulations.

In some embodiments, the correlating can comprise assessing cell numbers of the one subpopulation relative to one or more additional subpopulations of the at least two subpopulations. In some embodiments, the correlating can comprise assessing cell numbers of the one subpopulation relative to (i) cell numbers of the independently expected subpopulation and/or (ii) cell numbers of the dataset subpopulation.

In some embodiments, a trait of a cell (e.g., expected or known) can comprise presence or absence of one or more cellular components, as disclosed herein. Non-limiting examples of the at least one trait can include presence of a protein (e.g., a surface protein), a genetic allele, a biochemical trait (e.g., expression and/or activity level of one or more genes of interest), a response to a pharmaceutical, a transcriptome expression profile, mRNA expression level of one or more genes of interest, a proteome expression profile, and a protein expression or activity level.

In some embodiments, a cell that is partitioned may not arise from mitosis subsequent to or during the partitioning or once partitioned. In some embodiments, the partitioning of a cell from a pool of cells may not substantially change one or more characteristics (e.g., or traits) of the cell and analyses thereof. For example, the cell partitioning may not substantially change (e.g., decrease and/or increase) expression or activity level of a gene of interest in the cell. In another example, the cell partitioning may not substantially change transcriptional profile of the cell. In some cases, upon the cell partitioning as disclosed herein, a degree of change of one or more characteristics of the cell (e.g., as compared to that prior to the cell partitioning, or as compared to a control cell that is not subjected to such partitioning) may be less than or equal to about 20%, less than or equal to about 19%, less than or equal to about 18%, less than or equal to about 17%, less than or equal to about 16%, less than or equal to about 15%, less than or equal to about 14%, less than or equal to about 13%, less than or equal to about 12%, less than or equal to about 11%, less than or equal to about 10%, less than or equal to about 9%, less than or equal to about 8%, less than or equal to about 7%, less than or equal to about 6%, less than or equal to about 5%, less than or equal to about 4%, less than or equal to about 3%, less than or equal to about 2%, less than or equal to about 1%, less than or equal to about 0.9%, less than or equal to about 0.8%, less than or equal to about 0.7%, less than or equal to about 0.6%, less than or equal to about 0.5%, less than or equal to about 0.4%, less than or equal to about 0.3%, less than or equal to about 0.2%, or less than or equal to about 0.1. In some embodiments, any of the operations, steps, or methods disclosed herein can be processed or performed (e.g., automatically) in real-time. The term “real time” or “real-time,” as used interchangeably herein, generally refers to an event (e.g., an operation, a process, a method, a technique, a computation, a calculation, an analysis, an optimization, etc.) that is performed using recently obtained (e.g., collected or received) data. Examples of the event may include, but are not limited to, analysis of a one or more images of a cell to make a decision to partition the cell, correlating a partition to a data (e.g., imaging data or analysis thereof, a different data set, such as omics data, etc.), updating one or more deep learning algorithms (e.g., neural networks) for classification and sorting, controlling one or more process within the flow channel (e.g., actuation of one or more valves by at a sorting bifurcation, etc.) based on any analysis of the imaging of cells or the flow channel, etc. In some cases, a real time event may be performed almost immediately or within a short enough time span, such as within at least 0.0001 ms, 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05 ms, 0.01 ms, 0.005 ms, 0.001 ms, 0.0005 ms, 0.0001 ms, or less.

In some embodiments, the terms “sorting” (or “sort”) and “partitioning” (or “partition) may be used interchangeably. As such, as disclosed herein, a sorting module may function as a partitioning module.

### II. Cell Clustering Map

In some embodiments, cell imaging data (e.g., imaging data of cells with or without staining by the heterologous marker as disclosed herein) can be analyzed to plot (e.g., in silico) a plurality of cells into a cell clustering map. Image data of a plurality of cells can be obtained, wherein the image data comprises tag-free images of single cells. The image data can be processed to generate a cell morphology map (e.g., one or more cell morphology maps). The cell morphology map can comprise a plurality of morphologically distinct clusters corresponding to different types or states of the cells. In some cases, a classifier (e.g., a cell clustering machine learning algorithm or deep learning algorithm) can be trained by using the cell morphology map. In some cases, the classifier can be configured to classify (e.g., automatically classify) a cellular image sample based on its proximity, correlation, or commonality with one or more of the morphologically distinct clusters. Thus, in some cases, the classifier can be used to classify (e.g., automatically classify) the cellular image sample accordingly.

The term “cluster” as used herein generally refers to a group of datapoints, such that datapoints in one group (e.g., a first cluster) are more similar to each other than datapoints of another group (e.g., a second cluster). A cluster can be a group of like datapoints (e.g., each datapoint representing a cell or an image of a cell) that are grouped together based on the proximity of the datapoints, to a measure of central tendency of the cluster. For example, a population of cells can be analyzed based on one or more morphological properties of each cell (e.g., by analyzing one or more images of each cell), and each cell can be plotted as a datapoint on a map base on the one or more morphological properties of each cell. Following, one or more clusters comprising a plurality of datapoints based on the proximity of the datapoints. The central tendency of each cluster can be measured by one or more algorithms (e.g., hierarchical clustering models, K-means algorithm, statistical distribution models, etc.). For instance, the measure of central tendency may be the arithmetic mean of the cluster, in which case the datapoints are joined together based on their proximity to the average value in the cluster (e.g., K-means clustering), their correlation, or their commonality.

The term “classifier” as used herein generally refers to an analysis model (e.g., a metamodel) that can be trained by using a learning model and applying learning algorithms (e.g., machine learning algorithms) on a training dataset (e.g., a dataset comprising examples of specific classes). In some cases, given a set of training examples/cases, each marked for belonging to a specific class (e.g., specific cell type or class), a training algorithm can build a classifier model capable of assigning new examples/cases (e.g., new datapoints of a cell or a group of cells) into one category or the other, e.g., to make the model a non-probabilistic classifier. In some cases, the classifier model can be capable of creating a new category to assign new examples/cases into the new category. In some cases, a classifier model can be the actual trained classifier that is generated based on the training model.

The term “cell type” as used herein generally refers to a kind, identity, or classification of cells according to one or more criteria, such as a tissue and species of origin, a differentiation state, whether or not they are healthy/normal or diseased, cell cycle stage, viability, etc. In non-limiting examples, the term “cell type” can refer specifically to any specific kind of cell, such as an embryonic stem cell, a neural precursor cell, a myoblast, a mesodermal cell, etc.

The term “cell state” as used herein generally refers to a specific state of the cell, such as but not limited to an activated cell, such as activated neuron or immune cell, resting cell, such as a resting neuron or immune cell, a dividing cell, quiescent cell, or a cell during any stages of the cell cycle.

The term “cell cycle” as used herein generally refers to the physiological and/or morphological progression of changes that cells undergo when dividing (e.g., proliferating). Examples of different phases of the cell cycle can include “interphase,” “prophase,” “metaphase,” “anaphase,” and “telophase”. Additionally, parts of the cell cycle can be “M (mitosis),” “S (synthesis),” “GO,”“G1 (gap 1)” and “G2 (gap2)”. Furthermore, the cell cycle can include periods of progression that are intermediate to the above named phases.

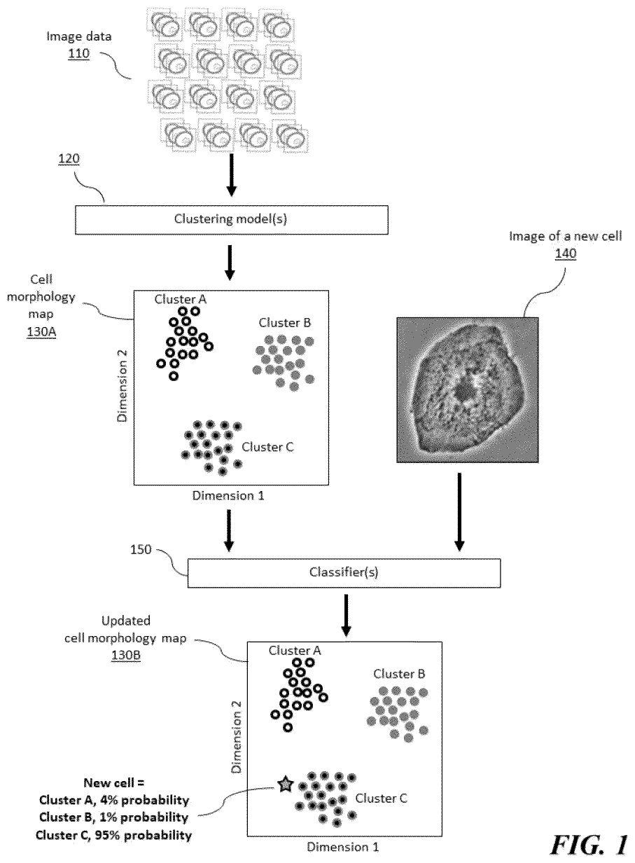

FIG. 1 schematically illustrates an example method for classifying a cell. The method can comprise processing image data 110 comprising tag-free images/videos of single cells (e.g., image data 110 consisting of tag-free images/videos of single cells). Various clustering analysis models 120 as disclosed herein can be used to process the image data 110 to extract one or more morphological properties of the cells from the image data 110, and generate a cell morphology map 130A based on the extracted one or more morphological properties. For example, the cell morphology map 130A can be generated based on two morphological properties as dimension 1 and dimension 2. The cell morphology map 130A can comprise one or more clusters (e.g., clusters A, B, and C) of datapoints, each datapoint representing an individual cell from the image data 110. The cell morphology map 130A and the clusters A-C therein can be used to train classifier(s) 150. Subsequently, a new image 140 of a new cell can be obtained and processed by the trained classifier(s) 150 to automatically extract and analyze one or more morphological features from the cellular image 140 and plot it as a datapoint on the cell morphology map 130A. Based on its proximity, correlation, or commonality with one or more of the morphologically-distinct clusters A-C on the cell morphology map 130A, the classifier(s) 150 can automatically classify the new cell. The classifier(s) 150 can determine a probability that the cell in the new image data 140 belongs to cluster C (e.g., the likelihood for the cell in the new image data 140 to share one or more commonalities and/or characteristics with cluster C more than with other clusters A/B). For example, the classifier(s) 150 can determine and report that the cell in the new image data 140 has a 95% probability of belonging to cluster C, 1% probability of belonging to cluster B, and 4% probability of belong to cluster A, solely based on analysis of the tag-free image 140 and one or more morphological features of the cell extracted therefrom.

An image and/or video (e.g., a plurality of images and/or videos) of one or more cells as disclosed herein (e.g., that of image data 110 in FIG. 1) can be captured while the cell(s) is suspended in a fluid (e.g., an aqueous liquid, such as a buffer) and/or while the cell(s) is moving (e.g., transported across a microfluidic channel). For example, the cell may not and need not be suspended is a gel-like or solid-like medium. The fluid can comprise a liquid that is heterologous to the cell(s)'s natural environment. For example, cells from a subject's blood can be suspended in a fluid that comprises (i) at least a portion of the blood and (ii) a buffer that is heterologous to the blood. The cell(s) may not be immobilized (e.g., embedded in a solid tissue or affixed to a microscope slide, such as a glass slide, for histology) or adhered to a substrate.

The cell(s) may be isolated from its natural environment or niche (e.g., a part of the tissue the cell(s) would be in if not retrieved from a subject by human intervention) when the image and/or video of the cell(s) is captured. For example, the image and/or video may not and need not be from a histological imaging. The cell(s) may not and need not be sliced or sectioned prior to obtaining the image and/or video of the cell, and, as such, the cell(s) may remain substantially intact as a whole during capturing of the image and/or video.

When the image data is processed, e.g., to extract one or more morphological features of a cell, each cell image may be annotated with the extracted one or more morphological features and/or with information that the cell image belongs to a particular cluster (e.g., a probability).

The cell morphology map can be a visual (e.g., graphical) representation of one or more clusters of datapoints. The cell morphology map can be a 1-dimensional (1D) representation (e.g., based on one morphological property as one parameter or dimension) or a multi-dimensional representation, such as a 2-dimensional (2D) representation (e.g., based on two morphological properties as two parameters or dimensions), a 3-dimensional (3D) representation (e.g., based on three morphological properties as three parameters or dimensions), a 4-dimensional (4D) representation, etc. In some cases, one morphological properties of a plurality of morphological properties used for blotting the cell morphology map can be represented as a non-axial parameter (e.g., non-x, y, or z axis), such as, distinguishable colors (e.g., heatmap), numbers, letters (e.g., texts of one or more languages), and/or symbols (e.g., a square, oval, triangle, square, etc.). For example, a heatmap can be used as colorimetric scale to represent the classifier prediction percentages for each cell against a cell class, cell type, or cell state.

The cell morphology map can be generated based on one or more morphological features (e.g., characteristics, profiles, fingerprints, etc.) from the processed image data. Non-limiting examples of one or more morphological properties of a cell, as disclosed herein, that can be extracted from one or more images of the cell can include, but are not limited to (i) shape, curvature, size (e.g., diameter, length, width, circumference), area, volume, texture, thickness, roundness, etc. of the cell or one or more components of the cell (e.g., cell membrane, nucleus, mitochondria, etc.), (ii) number or positioning of one or more contents (e.g., nucleus, mitochondria, etc.) of the cell within the cell (e.g., center, off-centered, etc.), and (iii) optical characteristics of a region of the image(s) (e.g., unique groups of pixels within the image(s)) that correspond to the cell or a portion thereof (e.g., light emission, transmission, reflectance, absorbance, fluorescence, luminescence, etc.).

Non-limiting examples of clustering as disclosed herein can be hard clustering (e.g., determining whether a cell belongs to a cluster or not), soft clustering (e.g., determining a likelihood that a cell belongs to each cluster to a certain degree), strict partitioning clustering (e.g., determining whether each cell belongs to exactly one cluster), strict partitioning clustering with outliers (e.g., determining whether a cell can also belong to no cluster), overlapping clustering (e.g., determining whether a cell can belong to more than one cluster), hierarchical clustering (e.g., determining whether cells that belong to a child cluster can also belong to a parent cluster), and subspace clustering (e.g., determining whether clusters are not expected to overlap).

Cell clustering and/or generation of the cell morphology map, as disclosed herein, can be based on a single morphological property of the cells. Alternatively, cell clustering and/or generation the cell morphology map can be based on a plurality of different morphological properties of the cells. In some cases, the plurality of different morphological properties of the cells can have the same weight or different weights. A weight can be a value indicative of the importance or influence of each morphological property relative to one another in training the classifier or using the classifier to (i) generate one or more cell clusters, (ii) generate the cell morphology map, or (iii) analyze a new cellular image to classify the cellular image as disclosed herein. For example, cell clustering can be performed by having 50% weight on cell shape, 40% weight on cell area, and 10% weight on texture (e.g., roughness) of the cell membrane. In some cases, the classifier as disclosed herein can be configured to adjust the weights of the plurality of different morphological properties of the cells during analysis of new cellular image data, thereby to yield a most optimal cell clustering and cell morphology map. The plurality of different morphological properties with different weights can be utilized during the same analysis step for cell clustering and/or generation of the cell morphology map.

The plurality of different morphological properties can be analyzed hierarchically. In some cases, a first morphological property can be used as a parameter to analyze image data of a plurality of cells to generate an initial set of clusters. Subsequently, a second and different morphological property can be used as a second parameter to (i) modify the initial set of clusters (e.g., optimize arrangement among the initial set of clusters, re-group some clusters of the initial set of clusters, etc.) and/or (ii) generate a plurality of sub-clusters within a cluster of the initial set of clusters. In some cases, a first morphological property can be used as a parameter to analyze image data of a plurality of cells to generate an initial set of clusters, to generate a 1D cell morphology map. Subsequently, a second morphological property can be used as a parameter to further analyze the clusters of the 1D cell morphology map, to modify the clusters and generate a 2D cell morphology map (e.g., a first axis parameter based on the first morphological property and a second axis parameter based on the second morphological property).

In some cases of the hierarchical clustering as disclosed herein, an initial set of clusters can be generated based on an initial morphological feature that is extracted from the image data, and one or more clusters of the initial set of clusters can comprise a plurality of sub-clusters based on second morphological features or sub-features of the initial morphological feature. For example, the initial morphological feature can be stem cells (or not), and the sub-features can be different types of stem cells (e.g., embryonic stem cells, induced pluripotent stem cells, mesenchymal stem cells, muscle stem cells, etc.). In another example, the initial morphological feature can be cancer cells (or not), and the sub-feature can be different types of cancer cells (e.g., sarcoma cells, sarcoma cells, leukemia cells, lymphoma cells, multiple myeloma cells, melanoma cells, etc.). In a different example, the initial morphological feature can be cancer cells (or not), and the sub-feature can be different stages of the cancer cell (e.g., quiescent, proliferative, apoptotic, etc.).

Each datapoint can represent an individual cell or a collection of a plurality of cells (e.g., at least or up to about 2, 3, 4, 5, 6, 7, 8, 9, or 10 cells). Each datapoint can represent an individual image (e.g., of a single cell or a plurality of cells) or a collection of a plurality of images (e.g., at least or up to about 2, 3, 4, 5, 6, 7, 8, 9, or 10 images of the same single cell or different cells).

The cell morphology map can comprise at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 15, at least or up to about 20, at least or up to about 30, at least or up to about 40, at least or up to about 50, at least or up to about 60, at least or up to about 70, at least or up to about 80, at least or up to about 90, at least or up to about 100, at least or up to about 150, at least or up to about 200, at least or up to about 300, at least or up to about 400, at least or up to about 500 clusters.

Each cluster as disclosed herein can comprise a plurality of sub-clusters, e.g., at least or up to about 2, at least or up to about 3, at least or up to about 4, at least or up to about 5, at least or up to about 6, at least or up to about 7, at least or up to about 8, at least or up to about 9, at least or up to about 10, at least or up to about 15, at least or up to about 20, at least or up to about 30, at least or up to about 40, at least or up to about 50, at least or up to about 60, at least or up to about 70, at least or up to about 80, at least or up to about 90, at least or up to about 100, at least or up to about 150, at least or up to about 200, at least or up to about 300, at least or up to about 400, at least or up to about 500 sub-clusters,

A cluster (or sub-cluster) can comprise datapoints representing cells of the same type/state. Alternatively, a cluster (or sub-cluster) can comprise datapoints representing cells of different types/states.

A cluster (or sub-cluster) can comprise at least or up to about 1, at least or up to about 2, at least or up to about 3, at least or up to about 4, at least or up to about 5, at least or up to about 6, at least or up to about 7, at least or up to about 8, at least or up to about 9, at least or up to about 10, at least or up to about 15, at least or up to about 20, at least or up to about 30, at least or up to about 40, at least or up to about 50, at least or up to about 60, at least or up to about 70, at least or up to about 80, at least or up to about 90, at least or up to about 100, at least or up to about 150, at least or up to about 200, at least or up to about 300, at least or up to about 400, at least or up to about 500, at least or up to about 1,000, at least or up to about 2,000, at least or up to about 3,000, at least or up to about 4,000, at least or up to about 5,000, at least or up to about 10000, at least or up to about 50,000, or at least or up to about 100,000 datapoints.

Two or more clusters may overlap in a cell morphology map. Alternatively, no clusters may not overlap in a cell morphology map. In some cases, an allowable degree of overlapping between two or more clusters may be adjustable (e.g., manually or automatically by a machine learning algorithm) depending on the quality, condition, or size of data in the image data being processed.

A cluster (or sub-cluster) as disclosed herein can be represented with a boundary (e.g., a solid line or a dashed line). Alternatively, a cluster or sub-cluster may not and need not be represented with a boundary, and may be distinguishable from other cluster(s) sub-cluster(s) based on their proximity to one another.

A cluster (or sub-cluster) or a data comprising information about the cluster can be annotated based on one or more annotation schema (e.g., predefined annotation schema). Such annotation can be manual (e.g., by a user of the method or system disclosed herein) or automatically (e.g., by any of the machine learning algorithms disclosed herein). The annotation of the clustering can be related the one or more morphological properties of the cells that have been analyzed (e.g., cell shape, cell area, optical characteristic(s), etc.) to generate the cluster or assign one or more datapoints to the cluster. Alternatively, the annotation of the clustering can be related to information that has not been used or analyzed to generate the cluster or assign one or more datapoints to the cluster (e.g., genomics, transcriptomics, or proteomics, etc.). In such case, the annotation can be utilized to add additional “layers”of information to each cluster.

In some cases, an interactive annotation tool can be provided that permits one or more users to modify any process of the method described herein. For example, the interactive annotation tool can allow a user to curate, verify, edit, and/or annotate the morphologically-distinct clusters. In another example, the interactive annotation tool can process the image data, extract one or more morphological features from the image data, and allow the user to select one or more of the extracted morphological features to be used as a basis to generate the clusters and/or the cell morphology map. After the generation of the clusters and/or the cell morphology map, the interactive annotation tool can allow the user to annotate each cluster and/or the cell morphology map using (i) a predefined annotation schema or (ii) a new, user-defined annotation schema. In another example, the interactive annotation tool can allow user to assign different weights to different morphological features for the clustering and/or map plotting. In another example, the interactive annotation tool can allow user to select with imaging data (or which cells) to be used and/or which imaging data (or which cells, cell clumps, artifacts, or debris) to be discarded, for the clustering and/or map plotting. A user can manually identify incorrectly clustered cells, or the machine learning algorithm can provide probability or correlation value of cells within each cluster and identify any outlier (e.g., a datapoint that would change the outcome of the probability/correlation value of the cluster(s) by a certain percentage value). Thus, the user can choose to move the outliers via the interactive annotation tool to further tune the cell morphology map, e.g., to yield a “higher resolution”map.

One or more cell morphology maps as disclosed herein can be used to train one or more classifiers (e.g., at least or up to about 1, 2, 3, 4, 5, 6, 7, 8, 9, 10, or more classifiers) as disclosed herein. Each classifier can be trained to analyze one or more images of a cell (e.g., to extract one or more morphological features of the cell) and categorize (or classify) the cell into one or more determined class or categories of a cell (e.g., based on a type of state of the cell). Alternatively, the classifier can be trained to create a new category to categorize (or classify) the cell into the new category, e.g., when determining that the cell is morphologically distinct than any pre-existing categories of other cells.

The machine learning algorithm as disclosed herein can be configured to extract one or more morphological feature of a cell from the image data of the cell. The machine learning algorithm can form a new data set based on the extracted morphological features, and the new data set may not and need not contain the original image data of the cell. In some examples, replicas of the original images in the image data can be stored in a database disclosed herein, e.g., prior to using any of the new images for training, e.g., to keep the integrity of the images of the image data. In some examples, processed images of the original images in the image data can be stored in a database disclosed herein during or subsequent to the classifier training. In some cases, any of the newly extracted morphological features as disclosed herein can be utilized as new molecular markers for a cell or population of cells of interest to the user. As cell analysis platform as disclosed herein can be operatively coupled to one or more databases comprising non-morphological data of cells processed (e.g., genomics data, transcriptomics data, proteomics data, metabolomics data), a selected population of cells exhibiting the newly extracted morphological feature(s) can be further analyzed by their non-morphological properties to identify proteins or genes of interest that are common in the selected population of cells but not in other cells, thereby determining such proteins or genes of interest to be new molecular markers that can be used to identify such selected population of cells.

In some cases, a classifier can be trained by applying machine learning algorithms on at least a portion of one or more cell morphology maps as disclosed herein as a training dataset. Non-limiting examples of machine learning algorithms for training a classifier can include supervised learning, unsupervised learning, semi-supervised learning, reinforcement learning, self-learning, feature learning, anomaly detection, association rules, etc. In some cases, a classifier can be trained by using one or more learning models on such training dataset. Non-limiting examples of learning models can include artificial neural networks (e.g., convolutional neural networks, U-net architecture neural network, etc.), backpropagation, boosting, decision trees, support vector machines, regression analysis, Bayesian networks, genetic algorithms, kernel estimators, conditional random field, random forest, ensembles of classifiers, minimum complexity machines (MCM), probably approximately correct learning (PACT), etc.

In some cases, the neural networks are designed by the modification of neural networks such as AlexNet, VGGNet, GoogLeNet, ResNet (residual networks), DenseNet, and Inception networks. In some examples, the enhanced neural networks are designed by modification of ResNet (e.g. ResNet 18, ResNet 34, ResNet 50, ResNet 101, and ResNet 152) or inception networks. In some aspects, the modification comprises a series of network surgery operations that are mainly carried out to improve including inference time and/or inference accuracy.

The machine learning algorithm as disclosed herein can utilize one or more clustering algorithms to determine that objects in the same cluster can be more similar (in one or more morphological features) to each other than those in other clusters. Non-limiting examples of the clustering algorithms can include, but are not limited to, connectivity models (e.g., hierarchical clustering), centroid models (e.g. K-means algorithm), distribution models (e.g., expectation-maximization algorithm), density models (e.g., density-based spatial clustering of applications with noise (DBSCAN), ordering points to identify the clustering structure (OPTICS)), subspace models (e.g., biclustering), group models, graph-based models (e.g., highly connected subgraphs (HCS) clustering algorithms), single graph models, and neural models (e.g., using unsupervised neural network). The machine learning algorithm can utilize a plurality of models, e.g., in equal weights or in different weights.

In some cases, unsupervised and self-supervised approaches can be used to expedite labeling of image data of cells. For the case of unsupervised, an embedding for a cell image can be generated. For example, the embedding can be a representation of the image in a space with reduced dimensions than the original image data. Such embeddings can be used to cluster images that are similar to one another. Thus, the labeler can be configured to batch-label the cells and increase the throughput as compared to manually labeling one or more cells.

In some cases, for the case of self-supervised learning, additional meta information (e.g., additional non-morphological information) about the sample (e.g., what disease is known or associated with the patient who provided the sample) can be used for labeling of image data of cells.

In some cases, embedding generation can use a neural net trained on predefined cell types. To generate the embeddings described herein, an intermediate layer of the neural net that is trained on predetermined image data (e.g., image data of known cell types and/or states) can be used. By providing enough diversity in image data/sample data to the trained model/classifier, this method can provide an accurate way to cluster future cells.

In some cases, embedding generation can use neural nets trained for different tasks. To generate the embeddings described herein, an intermediate layer of the neural net that is trained for a different task (e.g., a neural net that is trained on a canonical dataset such as ImageNet). Without wishing to be bound by theory, this can allow to focus on features that matter for image classification (e.g., edges and curves) while removing a bias that may otherwise be introduced in labeling the image data.

In some cases, autoencoders can be used for embedding generation. To generate the embeddings described herein, autoencoders can be used, in which the input and the output can be substantially the same image and the squeeze layer can be used to extract the embeddings. The squeeze layer can force the model to learn a smaller representation of the image, which smaller representation may have sufficient information to recreate the image (e.g., as the output).

In some cases, for clustering-based labeling of image data or cells, as disclosed herein, an expanding training data set can be used. With the expanding training data set, one or more revisions of labeling (e.g., manual relabeling) may be needed to, e.g., avoid the degradation of model performance due to the accumulated effect of mislabeled images. Such manual relabeling may be intractable on a large scale and ineffective when done on a random subset of the data. Thus, to systematically surface images for potential relabeling, for example, similar embedding-based clustering can be used to identify labeled images that may cluster with members of other classes. Such examples are likely to be enriched for incorrect or ambiguous labels, which can be removed (e.g., automatically or manually).

In some cases, adaptive image augmentation can be used. In order to make the models and classifiers disclosed herein more robust to artifacts in the image data, (1) one or more images with artifacts can be identified, and (2) such images identified with artifacts can be added to training pipeline (e.g., for training the model/classifier). Identifying the image(s) with artifacts can comprise: (1a) while imaging cells, one or more additional sections of the image frame can be cropped, which frame(s) being expected to contain just the background without any cell; (2a) the background image can be checked for any change in one or more characteristics (e.g., optical characteristics, such as brightness); and (3a) flagging/labeling one or more images that have such change in the characteristic(s). Adding the identified images to training pipeline can comprise: (2a) adding the one or more images that have been flagged/labeled as augmentation by first calculating an average feature of the changed characteristic(s) (e.g., the background median color); (2b) creating a delta image by subtracting the average feature from the image data (e.g., subtracting the median for each pixel of the image); and (3c) adding the delta image to the training pipeline.

One or more dimension of the cell morphology map can be represented by various approaches (e.g., dimensionality reduction approaches), such as, for example, principal component analysis (PCA), multidimensional scaling (MDS), t-distributed stochastic neighbor embedding (t-SNE), and uniform manifold approximation and projection (UMAP). For example, UMAP can be a machine learning technique for dimension reduction. UMAP can be constructed from a theoretical framework based in Riemannian geometry and algebraic topology. UMAP can be utilized for a practical scalable algorithm that applies to real world data, such as morphological properties of one or more cells.

The cell morphology map as disclosed herein can comprise an ontology of the one or more morphological features. The ontology can be an alternative medium to represent a relationship among various datapoints (e.g., each representing a cell) analyzed from an image data. For example, an ontology can be a data structure of information, in which nodes can be linked by edges. An edge can be used to define a relationship between two nodes. For example, a cell morphology map can comprise a cluster comprising sub-clusters, and the relationship between the cluster and the sub-clusters can be represented in an nodes/edges ontology (e.g., an edge can be used to describe the relationship as a subclass of, genus of, part of, stem cell of, differentiated from, progeny of, diseased state of, targets, recruits, interacts with, same tissue, different tissue, etc.).

In some cases, one-to-one morphology to genomics mapping can be utilized. An image of a single cell or images of multiple “similar looking” cells can be mapped to its/their molecular profile(s) (e.g., genomics, proteomics, transcriptomics, etc.). In some examples, classifier-based barcoding can be performed. Each sorting event (e.g., positive classifier) can push the sorted cell(s) into an individual well or droplet with a unique barcode (e.g., nucleic acid or small molecule barcode). The exact barcode(s) used for that individual classifier positive event can be recorded and tracked. Following, the cells can be lysed and molecularly analyzed together with the barcode(s). The result of the molecular analysis can then be mapped (e.g., one-to-one) to the image(s) of the individual (or ensemble of) sorted cell(s) captured while the cell(s) was/were flowing in the flow channel. In some examples, class-based sorting can be utilized. Cells that are classified in the same class based at least on their morphological features can be sorted into a single well or droplet with a pre-determined barcoded material, and the cells can be lysed, molecularly analyzed, then any molecular information can be used for the one-to-one mapping as disclosed herein.

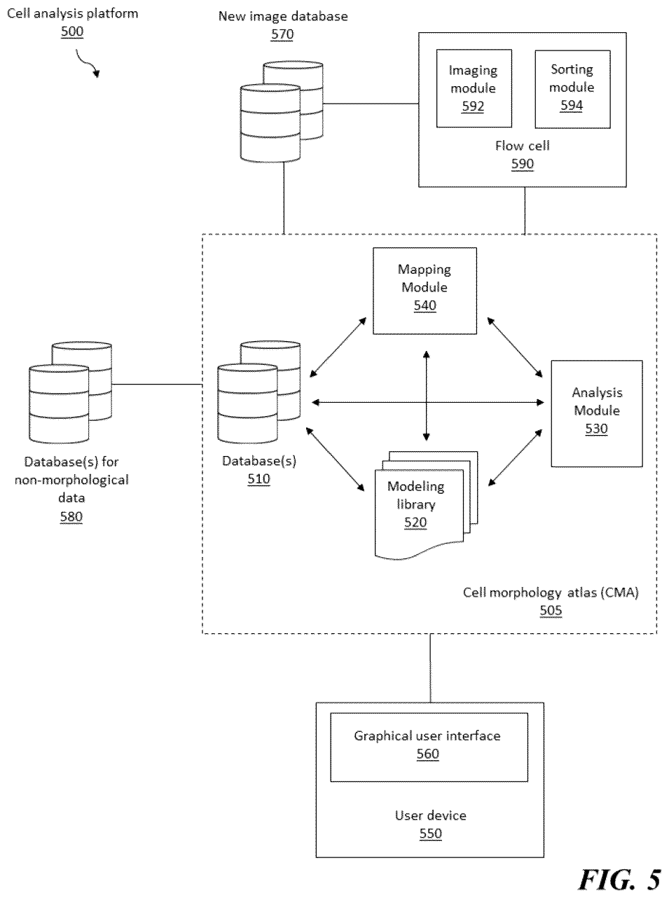

FIG. 5 illustrates an example cell analysis platform (e.g., machine learning/artificial intelligence platform) for analyzing image data of one or more cells. The cell analysis platform 500 can comprise a cell morphology atlas (CMA) 505. The CMA 505 can comprise a database 510 having a plurality of annotated single cell images that are grouped into morphologically-distinct clusters (e.g., represented a texts, as cell morphology map(s), or cell morphological ontology(ies)) corresponding to a plurality of classifications (e.g., predefined cell classes). The CMA 505 can comprise a modeling unit comprising one or more models (e.g., modeling library 520 comprising, such as, one or more machine learning algorithms disclosed herein) that are trained and validated using datasets from the CMA 505, to process image data comprising images/videos of one or more cells to identify different cell types and/or states based at least on morphological features. The CMA 505 can comprise an analysis module 530 comprising one or more classifiers as disclosed herein. The classifier(s) can uses one or more of the models from the modeling library 520 to, e.g., (1) classify one or more images taken from a sample, (2) assess a quality or state of the sample based on the one or more images, (3) map one or more datapoints representing such one or more images onto a cell morphology map (or cell morphological ontology) via using a mapping module 540. The CMA 505 can be operatively coupled to one or more additional database 570 to receive the image data comprising the images/videos of one or more cells. For example, the image data from the database 570 can be obtained from an imaging module 592 of a flow cell 590, which can also be operatively coupled to the CMA 505. The flow cell can direct flow of a sample comprising or suspected of comprising a target cell, and capture one or more images of contents (e.g., cells) within the sample by the imaging module 592. Any image data obtained by the imaging module 592 can be transmitted directly to the CMA 505 and/or to the new image database 570. Alternatively or in addition to, the CMA 505 can be operatively coupled to one or more additional databases 580 comprising non-morphological data of any of the cells (e.g., genomics, transcriptomics, or proteomics, etc.), e.g., to further annotate any of the datapoint, cluster, map, ontology, images, as disclosed herein. The CMA 505 can be operatively coupled to a user device 550 (e.g., a computer or a mobile device comprising a display) comprising a GUI 560 for the user to receive information from and/or to provide input (e.g., instructions to modify or assist any portion of the method disclosed herein). Any classification made by the CMA and/or the user can be provided as an input to the sorting module 594 of the flow cell 590. Based on the classification, the sorting module can determine, for example, (i) when to activate one or more sorting mechanisms at the sorting junction of the flow cell 590 to sort one or more cells of interest, (ii) which sub-channel of a plurality of sub-channels to direct each single cell for sorting. In some cases, the sorted cells can be collected for further analysis, e.g., downstream molecular assessment and/or profiling, such as genomics, transcriptomics, proteomics, metabolomics, etc.

Any of the methods or platforms disclosed herein can be used as a tool that permits a user to train one or more models (e.g., from the modeling library) for cell clustering and/or cell classification. For example, a user may provide initial image dataset of a sample to the platform, and the platform may process the initial set of image data. Based on the processing, the platform can determine a number of labels and/or an amount of data that the user needs to train the one or more models, based on the initial image dataset of the sample. In some examples, the platform can determine that the initial set of image data can be insufficient to provide an accurate cell classification or cell morphology map. For example, the platform can plot an initial cell morphology map and recommend to the user the number of labels and/or the amount of data needed to for enhanced processing, classification, and/or sorting, based on proximity (or separability), correlation, or commonality of the datapoints in the map (e.g., whether there is no distinguishable clusters within the map, whether the clusters within the map are too close to each other, etc.). In another example, the platform can allow the user to select different model (e.g., clustering model) or classifier, different combinations of models or classifiers, to re-analyze the initial set of image data.

Any of the methods or platforms disclosed herein can be used to determine quality or state of the image(s) of the cell, that of the cell, or that of a sample comprising the cell. The quality or state of the cell can be determined at a single cell level. Alternatively, the quality or state of the cell can be determined at an aggregate level (e.g., as a whole sample, or as a portion of the sample). The quality or state can be determined and reported based on, e.g., a number system (e.g., a number scale from 1 to 10, a percentage scale from 1% to 100%), a symbolic system, or a color system. For example, the quality or state can be indicative of a preparation or priming condition of the sample (e.g., whether the sample has a sufficient number of cells, whether the sample has too much artifacts, debris, etc.) or indicative of a viability of the sample (e.g., whether the sample has an amount of “dead”cells above a predetermined threshold).

Any of the methods or platforms disclosed herein can be used to sort cells in silico (e.g., prior to actual sorting of the cells using a microfluidic channel). The in silico sorting can be, e.g., to discriminate among and/or between, e.g., multiple different cell types (e.g., different types of cancer cells, different types of immune cells, etc.), cell states, cell qualities. The methods and platforms disclosed herein can utilize pre-determined morphological properties (e.g., provided in the platform) for the discrimination. Alternatively or in addition to, newly abstracted morphological properties can be abstracted (e.g., generated) based on the input data for the discrimination. In some cases, new model(s) and/or classifier(s) can be trained or generated to process the image data. In some cases, the newly abstracted morphological properties can be used to discriminate among and/or between, e.g., multiple different cell types, cell states, cell qualities that are known. Alternatively or in addition to, the newly abstracted morphological properties can be used to create new class (or classifications) to sort the cells (e.g., in silico or via the microfluidic system). The newly abstracted morphological properties as disclosed herein may enhance accuracy or sensitivity of cell sorting (e.g., in silico or via the microfluidic system).

Subsequent to the in silico sorting of the cells, the actual cell sorting of the cells (e.g., via the microfluidic system or flow cell) based on the in silico sorting can be performed within less than about 1 hours, 50 minutes, 40 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, 50 seconds, 40 seconds, 30 seconds, 20 seconds, 10 seconds, 5 seconds, 1 second, or less. In some cases, the in silico sorting and the actual sorting can occur in real-time.

In any of the methods or platforms disclosed herein, the model(s) and/or classifier(s) can be validated (e.g., for the ability to demonstrate accurate cell classification performance).

Non-limiting examples of validation metrics that can be utilized can include, but are not limited to, threshold metrics (e.g., accuracy, F-measure, Kappa, Macro-Average Accuracy, Mean-Class-Weighted Accuracy, Optimized Precision, Adjusted Geometric Mean, Balanced Accuracy, etc.), the ranking methods and metrics (e.g., receiver operating characteristics (ROC) analysis or “ROC area under the curve (ROC AUC)”), and the probabilistic metrics (e.g., root-mean-squared error). For example, the model(s) or classifier(s) can be determined to be balanced or accurate when the ROC AUC is greater than 0.5, greater than 0.55, greater than 0.6, greater than 0.65, greater than 0.7, greater than 0.75, greater than 0.8, greater than 0.85, greater than 0.9, greater than 0.91, greater than 0.92, greater than 0.93, greater than 0.94, greater than 0.95, greater than 0.96, greater than 0.97, greater than 0.98, greater than 0.99, or more.

In any of the methods or platforms disclosed herein, the image(s) of the cell(s) can be obtained when the cell(s) are prepared and diluted in a sample (e.g., a buffer sample). The cell(s) can be diluted, e.g., in comparison to real-life concentrations of the cell in the tissue (e.g., solid tissue, blood, serum, spinal fluid, urine, etc.) to a dilution concentration. The methods or platforms disclosed herein can be compatible with a sample (e.g., a biological sample or derivative thereof) that is diluted by a factor of about 500 to about 1,000,000. The methods or platforms disclosed herein can be compatible with a sample that is diluted by a factor of at least about 500. The methods or platforms disclosed herein can be compatible with a sample that is diluted by a factor of at most about 1,000,000. The methods or platforms disclosed herein can be compatible with a sample that is diluted by a factor of about 500 to about 1,000, about 500 to about 2,000, about 500 to about 5,000, about 500 to about 10,000, about 500 to about 20,000, about 500 to about 50,000, about 500 to about 100,000, about 500 to about 200,000, about 500 to about 500,000, about 500 to about 1,000,000, about 1,000 to about 2,000, about 1,000 to about 5,000, about 1,000 to about 10,000, about 1,000 to about 20,000, about 1,000 to about 50,000, about 1,000 to about 100,000, about 1,000 to about 200,000, about 1,000 to about 500,000, about 1,000 to about 1,000,000, about 2,000 to about 5,000, about 2,000 to about 10,000, about 2,000 to about 20,000, about 2,000 to about 50,000, about 2,000 to about 100,000, about 2,000 to about 200,000, about 2,000 to about 500,000, about 2,000 to about 1,000,000, about 5,000 to about 10,000, about 5,000 to about 20,000, about 5,000 to about 50,000, about 5,000 to about 100,000, about 5,000 to about 200,000, about 5,000 to about 500,000, about 5,000 to about 1,000,000, about 10,000 to about 20,000, about 10,000 to about 50,000, about 10,000 to about 100,000, about 10,000 to about 200,000, about 10,000 to about 500,000, about 10,000 to about 1,000,000, about 20,000 to about 50,000, about 20,000 to about 100,000, about 20,000 to about 200,000, about 20,000 to about 500,000, about 20,000 to about 1,000,000, about 50,000 to about 100,000, about 50,000 to about 200,000, about 50,000 to about 500,000, about 50,000 to about 1,000,000, about 100,000 to about 200,000, about 100,000 to about 500,000, about 100,000 to about 1,000,000, about 200,000 to about 500,000, about 200,000 to about 1,000,000, or about 500,000 to about 1,000,000. The methods or platforms disclosed herein can be compatible with a sample that is diluted by a factor of about 500, about 1,000, about 2,000, about 5,000, about 10,000, about 20,000, about 50,000, about 100,000, about 200,000, about 500,000, or about 1,000,000.

In any of the methods or platforms disclosed herein, the classifier can generate a prediction probability (e.g., based on the morphological clustering and analysis) that an individual cell or a cluster of cells belongs to a cell class (e.g., within a predetermined cell class provided in the CMA as disclosed herein), e.g., via a reporting module. The reporting module can communicate with the user via a GUI as disclosed herein. Alternatively or in addition to, the classifier can generate a prediction vector that an individual cell or a cluster of cells belongs to a plurality of cell classes (e.g., a plurality of all of predetermined cell classes from the CMA as disclosed herein). The vector can be 1D (e.g., a single row of different cell classes), 2D (e.g., two dimensions, such as tissue origin vs. cell type), 3D, etc. In some cases, based on processing and analysis of image data obtained from a sample, the classifier can generate a report showing a composition of the sample, e.g., a distribution of one or more cell types, each cell type indicated with a relative proportion within the sample. Each cell of the sample can also be annotated with a most probable cell type and one or more less probably cell types.

Any one of the methods and platforms disclosed herein can be capable of processing image data of one or more cells to generate one or more morphometric maps of the one or more cells. Non-limiting examples of morphometric models can be utilized to analyze one or more images of single cells (or cell clusters) can include, e.g., simple morphometrics (e.g., based on lengths, widths, masses, angles, ratios, areas, etc.), landmark-based geometric morphometrics (e.g., spatial information, intersections, etc. of one or more components of a cell), procrustes-based geometric morphometrics (e.g., by removing non-shape information that is altered by translation, scaling, and/or rotation from the image data), Euclidean distance matrix analysis, diffeomorphometry, and outline analysis. The morphometric map(s) can be multi-dimensional (e.g., 2D, 3D, etc.). The morphometric map(s) can be reported to the user via the GUI.

Any of the methods or platforms disclosed herein (e.g., the analysis module) can be used to process, analyze, classify, and/or compare two or more samples (e.g., at least 2, 3, 4, 5, 6, 7, 8, 9, 10, or more test samples). The two or more samples can each be analyzed to determine a morphological profile (e.g., a cell morphology map) of each sample. For example, the morphological profiles of the two or more samples can be compared for identifying a disease state of a patient's sample in comparison to a health cohort's sample or a sample of image data representative of a disease of interest. In another example, the morphological profiles of the two or more samples can be compared to monitor a progress of a condition of a subject, e.g., comparing first image data of a first set of cells from a subject before a treatment (e.g., a test drug candidate, chemotherapy, surgical resection of solid tumors, etc.) and second image data of a second set of cells from the subject after the treatment. The second set of cells can be obtained from the subject at least about 1 week, at least about 2 weeks, at least about 3 weeks, at least about 4 weeks, at least about 2 months, or at least about 3 months subsequent to obtaining the first set of cells from the subject. In a different example, the morphological profiles of the two or more samples can be compared to monitor effects of two or more different treatment options (e.g., different test drugs) in two or more different cohorts (e.g., human subjects, animal subjects, or cells being tested in vitro/ex vivo). Accordingly, the systems and methods disclosed herein can be utilized (e.g., via sorting or enrichment of a cell type of interest or a cell exhibiting a characteristic of interest) to select a drug and/or a therapy that yields a desired effect (e.g., a therapeutic effect greater than equal to a threshold value).

Any of the platforms disclosed herein (e.g., cell analysis platform) can provide an inline end-to-end pipeline solution for continuous labeling and/or sorting of multiple different cell types and/or states based at least in part on (e.g., based solely on) morphological analysis of imaging data provided. A modeling library used by the platform can be scalable for large amount of data, extensible (e.g., one or more models or classifiers modified), and/or generalizable (e.g., more resistant to data perturbations-such as artifacts, debris, random objects in the background, image/video distortions-between samples. Any of the modeling library may be removed or updated with new model automatically by the machine learning algorithms or artificial intelligence, or by the user.

Any of the methods and platforms disclosed herein can adjust one or more parameters of the microfluidic system as disclosed herein. As cells are flowing through a flow channel, an imaging module (e.g., sensors, cameras) can capture image(s)/video(s) of the cells and generate new image data. The image data can be processed and analyzed (e.g., in real-time) by the methods and platforms of the present disclosure to train a model (e.g., machine learning model) to determine whether or not one or more parameters of the microfluidic system.

In some cases, the model(s) can determine that the cells are flowing too fast or too slow, and send an instruction to the microfluidic system to adjust (i) the velocity of the cells (e.g., via adjusting velocity of the fluid medium carrying the cells) and/or (ii) image recording rate of a camera that is capturing images/videos of cells flowing through the flow channel.

In some cases, the model(s) can determine that the cells are in-focus or out-of-focus in the images/videos, and send an instruction to the microfluidic system to (i) adjust a positioning of the cells within the flow cell (e.g., move the cell towards or away from the center of the flow channel via, for example, hydrodynamic focusing and/or inertial focusing) and/or (ii) adjust a focal length/plane of the camera that is capturing images/videos of cells flowing through the flow channel. Adjusting the focal length/plane can be performed for the same cell that has been analyzed (e.g., adjusting focal length/plane of a camera that is downstream) or a subsequent cell. Adjusting the focal length/plane can enhance clarity or reduce blurriness in the images. The focal length/plane can be adjusted based on a classified type or state of the cell. In some examples, adjusting the focal length/plane can allow enhanced focusing/clarity on all parts of the cell. In some examples, adjusting the focal length/plane can allow enhanced focusing/clarity on different portions (but not all parts) of the cell. Without wishing to be bound by theory, out-of-focus images may be usable for any of the methods disclosed herein to extract morphological feature(s) of the cell that otherwise may not be abstracted from in-focus images, or vice versa. Thus, in some cases, instructing the imaging module to capture both in-focus and out-of-focus images of the cells can enhance accuracy of any of the analysis of cells disclosed herein. Alternatively or in addition to, the model(s) can send an instruction to the microfluidic system to modify the flow and adjust an angle of the cell relative to the camera, to adjust focus on different portions of the cell or a subsequent cell. Different portions as disclosed herein can comprise an upper portion, a mid portion, a lower portion, membrane, nucleus, mitochondria, etc. of the cell.

In some cases, the model(s) can determine that images of different modalities are needed for any of the analysis disclosed herein. Images of varying modalities can comprise a bright field image, a dark field image, a fluorescent image (e.g. of cells stained with a dye), an in-focus image, an out-of-focus image, a greyscale image, a monochrome image, a multi-chrome image, etc.

Any of the models or classifiers disclosed herein can be trained on a set of image data that is annotated with one imaging modality. Alternatively, the models/classifiers can be trained on set of image data that is annotated with a plurality of different imaging modalities (e.g., 2, 3, 4, 5, or more different imaging modalities). Any of the models/classifiers disclosed herein can be trained on a set of image data that is annotated with a spatial coordinate indicative of a position or location within the flow channel. Any of the models/classifiers disclosed herein can be trained on a set of image data that is annotated with a timestamp, such that a set of images can be processed based on the time they are taken.

An image of the image data can be processed in various image processing methods, such as horizontal or vertical image flips, orthogonal rotation, gaussian noise, contrast variation, or noise introduction to mimic microscopic particles or pixel-level aberrations. One or more of the processing methods can be used to generate replicas of the image or analyze the image. In some cases, the image can be processed into a lower-resolution image or a lower-dimension image (e.g., by using one or more deconvolution algorithm).

In any of the methods disclosed herein, processing an image or video from image data can comprise identifying, accounting for, and/or excluding one or more artifacts from the image/video, either automatically or manually by a user. Upon identification, the artifact(s) can be fed into any of the models or classifiers, to train image processing or image analysis. The artifact(s) can be accounted for when classifying the type or state of one or more cells in the image/video. The artifact(s) can be excluded from any determination of the type or state of the cell(s) in the image/video. The artifact(s) can be removed in silico by any of the models/classifiers disclosed herein, and any new replica or modified variant of the image/video excluding the artifact(s) can be stored in a database as disclosed herein. The artifact(s) can be, for example, from debris (e.g., dead cells, dust, etc.), optical conditions during capturing the image/video of the cells (e.g., lighting variability, over-saturation, under-exposure, degradation of the light source, etc.), external factors (e.g., vibrations, misalignment of the microfluidic chip relative to the lighting or optical sensor/camera, power surges/fluctuations, etc.), and changes to the microfluidic system (e.g., deformation/shrinkage/expansion of the microfluidic channel or the microfluidic chip as a whole). The artifacts can be known. The artifacts can be unknown, and the models or classifiers disclosed herein can be configured to define one or more parameters of a new artifact, such that the new artifact can be identified, accounted for, and/or excluded in image processing and analysis.

In some cases, a plurality of artifacts disclosed herein can be identified, accounted for, and/or excluded during image/video processing or analysis. The plurality of artifacts can be weighted the same (e.g., determined to have the same degree of influence on the image/video processing or analysis) or can have different weights (e.g., determined to have different degrees of influence on the image/video processing or analysis). Weight assignments to the plurality of artifacts can be instructed manually by the user or determined automatically by the models/classifiers disclosed herein.

In some cases, one or more reference images or videos of the flow channel (e.g., with or without any cell) can be stored in a database and used as a frame of reference to help identify, account for, and/or exclude any artifact. The reference image(s)/video(s) can be obtained before use of the microfluidic system. The reference image(s)/video(s) can be obtained during the use of the microfluidic system. The reference image(s)/video(s) can be obtained periodically during the use of the microfluidic system.

Any of the methods or platforms disclosed herein can be operatively coupled to an online crowdsourcing platform. The online crowdsourcing platform can comprise any of the database disclosed herein. For example, the database can store a plurality of single cell images that are grouped into morphologically-distinct clusters corresponding to a plurality of cell classes (e.g., predetermined cell types or states). The online crowdsourcing platform can comprise one or more models or classifiers as disclosed herein (e.g., a modeling library comprising one or more machine learning models/classifiers as disclosed herein). The online crowdsourcing platform can comprise a web portal for a community of users to share contents, e.g., (1) upload, download, search, curate, annotate, or edit one or more existing images or new images into the database, (2) train or validate the one or more model(s)/classifier(s) using datasets from the database, and/or (3) upload new models into the modeling library. In some cases, the online crowdsourcing platform can allow users to buy, sell, share, or exchange the model(s)/classifier(s) with one another.

In some cases, the web portal can be configured to generate incentives for the users to update the database with new annotated cell images, model(s), and/or classifier(s). Incentives may be monetary. Incentives may be additional access to the global CMA, model(s), and/or classifier(s). In some cases, the web portal can be configured to generate incentives for the users to download, use, and review (e.g., rate or leave comments) any of the annotated cell images, model(s), and/or classifier(s) from, e.g., other users.

In some cases, a global cell morphology atlas (global CMA) can be generated by collecting (i) annotated cell images, (ii) cell morphology maps or ontologies, (iii), and/or (iv) classifiers from the users via the web portal. The global CMA can then be shared with the users via the web portal. All users can have access to the global CMA. Alternatively, specifically defined users can have access to specifically defined portions of the global CMA. For example, cancer centers can have access to “cancer cells” portion of the global CMA, e.g., via a subscription based service. In a similar fashion, global models or classifiers may be generated based on the annotated cell images, model(s), and/or classifiers that are collected from the users via the web portal.

### III. Additional Aspects of Cell Analysis

Any of the systems and methods disclosed can be utilized to analyze a cell and/or sort (or partition) the cell from a population of cells. A cell may be directed through a flow channel, and one or more imaging devices (e.g., sensor(s), camera(s)) can be configured to capture one or more images/videos of the cell passing through. Subsequently, the image(s)/video(s) of the cell can be analyzed as disclosed herein (e.g., by the classifier to plot the cell as a datapoint in a cell morphology map, determine a most likely cluster it belongs to, and determine a final classification of the cell based on the selected cluster) in real-time, such that a decision can be made in real-time (e.g., automatically by the machine learning algorithm) to determine (i) whether to sort the cell or not and/or (ii) which sub-channel of a plurality of sub-channels to sort the cell into.

The cell sorting system as disclosed herein can comprise a flow channel configured to transport a cell through the channel. The cell sorting system can comprise an imaging device configured to capture an image of the cell from a plurality of different angles as the cell is transported through the flow channel. The cell sorting system can comprise a processor configured to analyze the image using a deep learning algorithm to enable sorting of the cell. The cell sorting system can be a cell classification system. In some cases, the flow channel can be configured to transport a solvent (e.g., liquid, water, media, alcohol, etc.) without any cell. The cell sorting system can have one or more mechanisms (e.g., a motor) for moving the imaging device relative to the channel. Such movement can be relative movement, and thus the moving piece can be the imaging device, the channel, or both. The processor can be further configured to control such relative movement.

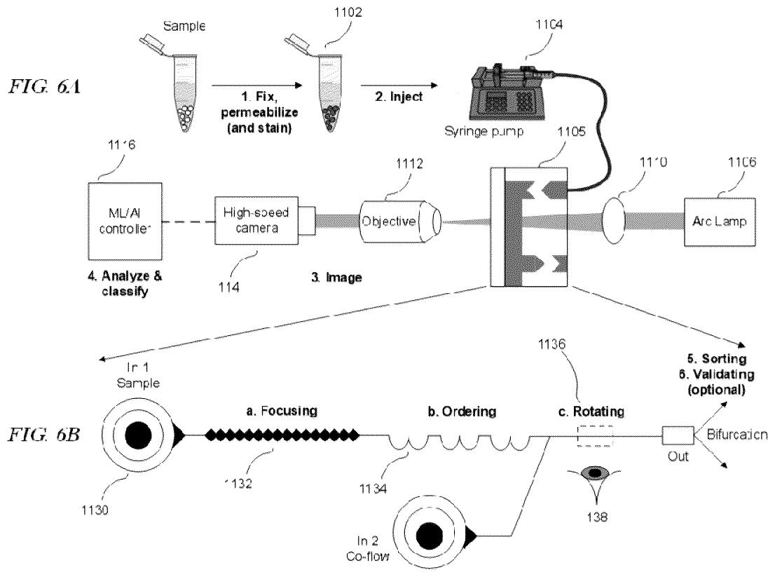

FIG. 6A shows a schematic illustration of the cell sorting system, as disclosed herein, with a flow cell design (e.g., a microfluidic design), with further details illustrated in FIG. 6B. The cell sorting system can be operatively coupled to a machine learning or artificial intelligence controller. Such ML/AI controller can be configured to perform any of the methods disclosed herein. Such ML/AI controller can be operatively coupled to any of the platforms disclosed herein.

In operation, a sample 1102 is prepared and injected by a pump 1104 (e.g., a syringe pump) into a flow cell 1105, or flow-through device. In some embodiments, the flow cell 1105 is a microfluidic device. Although FIG. 6A illustrates a classification and/or sorting system utilizing a syringe pump, any of a number of perfusion systems can be used such as (but not limited to) gravity feeds, peristalsis, or any of a number of pressure systems. In some embodiments, the sample is prepared by fixation and staining. In some examples, the sample comprises live cells. As can readily be appreciated, the specific manner in which the sample is prepared is largely dependent upon the requirements of a specific application.

Examples of the flow unit may be, but are not limited to, a syringe pump, a vacuum pump, an actuator (e.g., linear, pneumatic, hydraulic, etc.), a compressor, or any other suitable device to exert pressure (positive, negative, alternating thereof, etc.) to a fluid that may or may not comprise one or more particles (e.g., one or more cells to be classified, sorted, and/or analyzed). The flow unit may be configured to raise, compress, move, and/or transfer fluid into or away from the microfluidic channel. In some examples, the flow unit may be configured to deliver positive pressure, alternating positive pressure and vacuum pressure, negative pressure, alternating negative pressure and vacuum pressure, and/or only vacuum pressure. The flow cell of the present disclosure may comprise at least 1, 2, 3, 4, 5, 6, 7, 8, 9, 10, or more flow units. The flow cell may comprise at most 10, 9, 8, 7, 6, 5, 4, 3, 2, or 1 flow unit.

Each flow unit may be in fluid communication with at least 1, 2, 3, 4, 5, 6, 7, 8, 9, 10, or more sources of fluid. Each flow unit may be in fluid communication with at most 10, 9, 8, 7, 6, 5, 4, 3, 2, or 1 fluid. The fluid may contain the particles (e.g., cells). Alternatively, the fluid may be particle-free. The flow unit may be configured to maintain, increase, and/or decrease a flow velocity of the fluid within the microfluidic channel of the flow unit. Thus, the flow unit may be configured to maintain, increase, and/or decrease a flow velocity (e.g., downstream of the microfluidic channel) of the particles. The flow unit may be configured to accelerate or decelerate a flow velocity of the fluid within the microfluidic channel of the flow unit, thereby accelerating or decelerating a flow velocity of the particles.

The fluid may be liquid or gas (e.g., air, argon, nitrogen, etc.). The liquid may be an aqueous solution (e.g., water, buffer, saline, etc.). Alternatively, the liquid may be oil. In some cases, only one or more aqueous solutions may be directed through the microfluidic channels. Alternatively, only one or more oils may be directed through the microfluidic channels. In another alternative, both aqueous solution(s) and oil(s) may be directed through the microfluidic channels. In some examples, (i) the aqueous solution may form droplets (e.g., emulsions containing the particles) that are suspended in the oil, or (ii) the oil may form droplets (e.g., emulsions containing the particles) that are suspended in the aqueous solution.

As can readily be appreciated, any perfusion system, including but not limited to peristalsis systems and gravity feeds, appropriate to a given classification and/or sorting system can be utilized.

As noted above, the flow cell 1105 can be implemented as a fluidic device that focuses cells from the sample into a single streamline that is imaged continuously. In the illustrated embodiment, the cell line is illuminated by a light source 1106 (e.g., a lamp, such as an arc lamp) and an optical system 1110 that directs light onto an imaging region 1138 of the flow cell 1105. An objective lens system 1112 magnifies the cells by directing light toward the sensor of a high-speed camera system 114.

In some embodiments, a 10×, 20×, 40×, 60×, 80×, 100×, or 200× objective is used to magnify the cells. In some embodiments, a 10×, objective is used to magnify the cells. In some embodiments, a 20× objective is used to magnify the cells. In some embodiments, a 40× objective is used to magnify the cells. In some embodiments, a 60× objective is used to magnify the cells. In some embodiments, a 80× objective is used to magnify the cells. In some embodiments, a 100× objective is used to magnify the cells. In some embodiments, a 200× objective is used to magnify the cells. In some embodiments, a 10× to a 200× objective is used to magnify the cells, for example a 10×-20×, a 10×-40×, a 10×-60×, a 10×-80×, or a 10×-100× objective is used to magnify the cells.

As can readily be appreciated by a person having ordinary skill in the art, the specific magnification utilized can vary greatly and is largely dependent upon the requirements of a given imaging system and cell types of interest.

In some embodiments, one or more imaging devices may be used to capture images of the cell. In some aspects, the imaging device is a high-speed camera. In some aspects, the imaging device is a high-speed camera with a micro-second exposure time. In some instances, the exposure time is 1 millisecond. In some instances, the exposure time is between 1 millisecond (ms) and 0.75 millisecond. In some instances, the exposure time is between 1 ms and 0.50 ms. In some instances, the exposure time is between 1 ms and 0.25 ms. In some instances, the exposure time is between 0.75 ms and 0.50 ms. In some instances, the exposure time is between 0.75 ms and 0.25 ms. In some instances, the exposure time is between 0.50 ms and 0.25 ms. In some instances, the exposure time is between 0.25 ms and 0.1 ms. In some instances, the exposure time is between 0.1 ms and 0.01 ms. In some instances, the exposure time is between 0.1 ms and 0.001 ms. In some instances, the exposure time is between 0.1 ms and 1 microsecond (μs). In some aspects, the exposure time is between 1 μs and 0.1 μs. In some aspects, the exposure time is between 1 μs and 0.01 μs. In some aspects, the exposure time is between 0.1 μs and 0.01 μs. In some aspects, the exposure time is between 1 μs and 0.001 μs. In some aspects, the exposure time is between 0.1 μs and 0.001 μs. In some aspects, the exposure time is between 0.01 μs and 0.001 μs.

In some cases, the flow cell 1105 may comprise at least 1, 2, 3, 4, 5, 6, 7, 8, 9, 10, or more imaging devices (e.g., the high-speed camera system 114) on or adjacent to the imaging region 1138. In some cases, the flow cell may comprise at most 10, 9, 8, 7, 6, 5, 4, 3, 2, or 1 imaging device on or adjacent to the imaging region 1138. In some cases, the flow cell 1105 may comprise a plurality of imaging devices. Each of the plurality of imaging devices may use light from a same light source. Alternatively, each of the plurality of imaging devices may use light from different light sources. The plurality of imaging devices may be configured in parallel and/or in series with respect to one another. The plurality of imaging devices may be configured on one or more sides (e.g., two adjacent sides or two opposite sides) of the flow cell 1105. The plurality of imaging devices may be configured to view the imaging region 1138 along a same axis or different axes with respect to (i) a length of the flow cell 1105 (e.g., a length of a straight channel of the flow cell 1105) or (ii) a direction of migration of one or more particles (e.g., one or more cells) in the flow cell 1105.

One or more imaging devices of the present disclosure may be stationary while imaging one or more cells, e.g., at the imaging region 1138. Alternatively, one or more imaging devices may move with respect to the flow channel (e.g., along the length of the flow channel, towards and/or away from the flow channel, tangentially about the circumference of the flow channel, etc.) while imaging the one or more cells. In some examples, the one or more imaging devices may be operatively coupled to one or more actuators, such as, for example, a stepper actuator, linear actuator, hydraulic actuator, pneumatic actuator, electric actuator, magnetic actuator, and mechanical actuator (e.g., rack and pinion, chains, etc.).

In some cases, the flow cell 1105 may comprise at least 1, 2, 3, 4, 5, 6, 7, 8, 9, 10, or more imaging regions (e.g., the imaging region 1138). In some cases, the flow cell 1105 may comprise at most 10, 9, 8, 7, 6, 5, 4, 3, 2, or 1 imaging region. In some examples, the flow cell 1115 may comprise a plurality of imaging regions, and the plurality of imaging regions may be configured in parallel and/or in series with respect to each another. The plurality of imaging regions may or may not be in fluid communication with each other. In an example, a first imaging region and a second imaging region may be configured in parallel, such that a first fluid that passes through the first imaging region does not pass through a second imaging region. In another example, a first imaging region and a second imaging region may be configured in series, such that a first fluid that passes through the first imaging region also passes through the second imaging region.

The imaging device(s) (e.g., the high-speed camera) of the imaging system can comprise an electromagnetic radiation sensor (e.g., IR sensor, color sensor, etc.) that detects at least a portion of the electromagnetic radiation that is reflected by and/or transmitted from the flow cell or any content (e.g., the cell) in the flow cell. The imaging device can be in operative communication with one or more sources (e.g., at least 1, 2, 3, 4, 5, or more) of the electromagnetic radiation. The electromagnetic radiation can comprise one or more wavelengths from the electromagnetic spectrum including, but not limited to x-rays (about 0.1 nanometers (nm) to about 10.0 nm; or about 10¹⁸ Hertz (Hz) to about 10¹⁶ Hz), ultraviolet (UV) rays (about 10.0 nm to about 380 nm; or about 8×10¹⁶ Hz to about 10¹⁵ Hz), visible light (about 380 nm to about 750 nm; or about 8×10¹⁴ Hz to about 4×10¹⁴ Hz), infrared (IR) light (about 750 nm to about 0.1 centimeters (cm); or about 4×10¹⁴ Hz to about 5×10¹¹ Hz), and microwaves (about 0.1 cm to about 100 cm; or about 10⁸ Hz to about 5×10¹¹ Hz). In some cases, the source(s) of the electromagnetic radiation can be ambient light, and thus the cell sorting system may not have an additional source of the electromagnetic radiation.

The imaging device(s) can be configured to take a two-dimensional image (e.g., one or more pixels) of the cell and/or a three-dimensional image (e.g., one or more voxels) of the cell.

As can readily be appreciated, the exposure times can differ across different systems and can largely be dependent upon the requirements of a given application or the limitations of a given system such as but not limited to flow rates. Images are acquired and can be analyzed using an image analysis algorithm.

In some embodiments, the images are acquired and analyzed post-capture. In some aspects, the images are acquired and analyzed in real-time continuously. Using object tracking software, single cells can be detected and tracked while in the field of view of the camera. Background subtraction can then be performed. In a number of embodiments, the flow cell 1106 causes the cells to rotate as they are imaged, and multiple images of each cell are provided to a computing system 1116 for analysis. In some embodiments, the multiple images comprise images from a plurality of cell angles.

The flow rate and channel dimensions can be determined to obtain multiple images of the same cell from a plurality of different angles (i.e., a plurality of cell angles). A degree of rotation between an angle to the next angle may be uniform or non-uniform. In some examples, a full 360° view of the cell is captured. In some embodiments, 4 images are provided in which the cell rotates 90° between successive frames. In some embodiments, 8 images are provided in which the cell rotates 45° between successive frames. In some embodiments, 24 images are provided in which the cell rotates 15° between successive frames. In some embodiments, at least three or more images are provided in which the cell rotates at a first angle between a first frame and a second frame, and the cell rotates at a second angle between the second frame and a third frame, wherein the first and second angles are different. In some examples, less than the full 360° view of the cell may be captured, and a resulting plurality of images of the same cell may be sufficient to classify the cell (e.g., determine a specific type of the cell).

The cell can have a plurality of sides. The plurality of sides of the cell can be defined with respect to a direction of the transport (flow) of the cell through the channel. In some cases, the cell can comprise a stop side, a bottom side that is opposite the top side, a front side (e.g., the side towards the direction of the flow of the cell), a rear side opposite the front side, a left side, and/or a right side opposite the left side. In some cases, the image of the cell can comprise a plurality of images captured from the plurality of angles, wherein the plurality of images comprise: (1) an image captured from the top side of the cell, (2) an image captured from the bottom side of the cell, (3) an image captured from the front side of the cell, (4) an image captured from the rear side of the cell, (5) an image captured from the left side of the cell, and/or (6) an image captured from the right side of the cell.

In some embodiments, a two-dimensional “hologram” of a cell can be generated by superimposing the multiple images of the individual cell. The “hologram” can be analyzed to automatically classify characteristics of the cell based upon features including but not limited to the morphological features of the cell.

In some embodiments, 1, 2, 3, 4, 5, 6, 7, 8, 9, or 10 images are captured for each cell. In some embodiments, 5 or more images are captured for each cell. In some embodiments, from 5 to 10 images are captured for each cell. In some embodiments, 10 or more images are captured for each cell. In some embodiments, from 10 to 20 images are captured for each cell. In some embodiments, 20 or more images are captured for each cell. In some embodiments, from 20 to 50 images are captured for each cell. In some embodiments, 50 or more images are captured for each cell. In some embodiments, from 50 to 100 images are captured for each cell. In some embodiments, 100 or more images are captured for each cell. In some cases, at least 1, 2, 3, 4, 5, 6, 7, 8, 9, 10, 15, 20, 30, 40, 50, or more images may be captured for each cell at a plurality of different angles. In some cases, at most 50, 40, 30, 20, 15, 10, 9, 8, 7, 6, 5, 4, 3, or 2 images may be captured for each cell at a plurality of different angles.

In some embodiments, the imaging device is moved so as to capture multiple images of the cell from a plurality of angles. In some aspects, the images are captured at an angle between 0 and 90 degrees to the horizontal axis. In some aspects, the images are captured at an angle between 90 and 180 degrees to the horizontal axis. In some aspects, the images are captured at an angle between 180 and 270 degrees to the horizontal axis. In some aspects, the images are captured at an angle between 270 and 360 degrees to the horizontal axis.

In some embodiments, multiple imaging devices (for e.g. multiple cameras) are used wherein each device captures an image of the cell from a specific cell angle. In some aspects, 2, 3, 4, 5, 6, 7, 8, 9, or 10 cameras are used. In some aspects, more than 10 cameras are used, wherein each camera images the cell from a specific cell angle, As can readily be appreciated, the number of images that are captured is dependent upon the requirements of a given application or the limitations of a given system. In several embodiments, the flow cell has different regions to focus, order, and/or rotate cells. Although the focusing regions, ordering regions, and cell rotating regions are discussed as affecting the sample in a specific sequence, a person having ordinary skill in the art would appreciate that the various regions can be arranged differently, where the focusing, ordering, and/or rotating of the cells in the sample can be performed in any order. Regions within a microfluidic device implemented in accordance with an embodiment of the disclosure are illustrated in FIG. 6B. Flow cell 1105 may include a filtration region 1130 to prevent channel clogging by aggregates/debris or dust particles. Cells pass through a focusing region 1132 that focuses the cells into a single streamline of cells that are then spaced by an ordering region 1134. In some embodiments, the focusing region utilizes “inertial focusing” to form the single streamline of cells. In some embodiments, the focusing region utilizes ‘hydrodynamic focusing” to focus the cells into the single streamline of cells. Optionally, prior to imaging, rotation can be imparted upon the cells by a rotation region 1136. The optionally spinning cells can then pass through an imaging region 1138 in which the cells are illuminated for imaging prior to exiting the flow cell. These various regions are described and discussed in further detail below. In some cases, the rotation region 1136 may precede the imaging region 1138. In some cases, the rotation region 1136 may be a part (e.g., a beginning portion, a middle portion, and/or an end portion with respect to a migration of a cell within the flow cell) of the imaging region 1138. In some cases, the imaging region 1138 may be a part of the rotation region 1136.

In some cases, only a single cell may be allowed to be transported across a cross-section of the flow channel perpendicular to the axis of the flow channel. In some cases, a plurality of cells (e.g., at least 2, 3, 4, 5, or more cells; at most 5, 4, 3, 2, or 1 cell) may be allowed to be transported simultaneously across the cross-section of the flow channel perpendicular to the axis of the flow channel. In such a case, the imaging device (or the processor operatively linked to the imaging device) may be configured to track each of the plurality of cells as they are transported along the flow channel.

The imaging system can include, among other things, a camera, an objective lens system and a light source. In a number of embodiments, flow cells similar to those described above can be fabricated using standard 2D microfluidic fabrication techniques, requiring minimal fabrication time and cost.

Although specific classification and/or sorting systems, flow cells, and microfluidic devices are described above with respect to FIGS. 6A and 6B, classification and/or sorting systems can be implemented in any of a variety of ways appropriate to the requirements of specific applications in accordance with various embodiments of the disclosure. Specific elements of microfluidic devices that can be utilized in classification and/or sorting systems in accordance with some embodiments of the disclosure are discussed further below.

In some cases, embodiments, the microfluidic system can comprise a microfluidic chip (e.g., comprising one or more microfluidic channels for flowing cells) operatively coupled to an imaging device (e.g., one or more cameras). A microfluidic device can comprise the imaging device, and the chip can be inserted into the device, to align the imaging device to an imaging region of a channel of the chip. To align the chip to the precise location for the imaging, the chip can comprise one or more positioning identifiers (e.g., pattern(s), such as numbers, letters, symbols, or other drawings) that can be imaged to determine the positioning of the chip (and thus the imaging region of the channel of the chip) relative to the device as a whole or relative to the imaging device. For image-based alignment (e.g., auto-alignment) of the chip within the device, one or more images of the chip can be capture upon its coupling to the device, and the image(s) can be analyzed by any of the methods disclosed herein (e.g., using any model or classifier disclosed herein) to determine a degree or score of chip alignment. The positioning identifier(s) can be a “guide” to navigate the stage holding the chip within the device to move within the device towards a correct position relative to the imaging unit.

In some cases, rule-based image processing can be used to navigate the stage to a precise range of location or a precise location relative to the image unit.

In some cases, machine learning/artificial intelligence methods as disclosed herein can be modified or trained to identify the pattern on the chip and navigate the stage to the precise imaging location for the image unit, to increase resilience.

In some cases, machine learning/artificial intelligence methods as disclosed herein can be modified or trained to implement reinforcement learning based alignment and focusing. The alignment process for the chip to the instrument or the image unit can involve moving the stage holding the chip in, e.g., either X or Y axis and/or moving the imaging plane on the Z axis. In the training process, (i) the chip can start at a X, Y, and Z position (e.g., randomly selected), (ii) based on one or more image(s) of the chip and/or the stage holding the chip, a model can determine a movement vector for the stage and a movement for the imaging plane, (iii) depending on whether such movement vector may take the chip closer to the optimum X, Y, and Z position relative to the image unit, an error term can be determined as a loss for the model, and (iv) the magnitude of the error can be either constant or be proportional to how far the current X, Y, and Z position is from an optimal X, Y, and Z position (e.g., may be predetermined). Such trained model can be used to determine, for example, the movement vector and/or movement of the movement for the imaging plane, to enhance relative alignment between the chip and the image unit (e.g., one or more sensors).

The alignment can occur subsequent to capturing of the image(s). Alternatively or in addition to, the alignment can occur real-time while capturing images/videos of the positioning identifier(s) of the chip.

One or more flow channels of the flow cell of the present disclosure may have various shapes and sizes. For example, referring to FIGS. 6A and 6B, at least a portion of the flow channel (e.g., the focusing region 1132, the ordering region 1134, the rotation region 1136, the imaging region 1138, connecting region therebetween, etc.) may have a cross-section that is circular, triangular, square, rectangular, pentagonal, hexagonal, or any partial shape or combination of shapes thereof.

### Cell Rotating Regions and Imaging Regions

Architecture of the microfluidic channel of the flow cell of the present disclosure may be controlled (e.g., modified, optimized, etc.) to modulate cell flow along the microfluidic channels. Examples of the cell flow may include (i) cell focusing (e.g., into a single streamline) and (ii) rotation of the at least one cell (or the one or more cells) as the cell(s) are migrating (e.g., within the single streamline) down the length of the microfluidic channels. In some embodiments, microfluidic channels can be configured to impart rotation on ordered cells in accordance with a number of embodiments of the disclosure. One or more cell rotation regions (e.g., the cell rotation region 1136) of microfluidic channels in accordance with some embodiments of the disclosure use co-flow of a particle-free buffer to induce cell rotation by using the co-flow to apply differential velocity gradients across the cells. In some cases, a cell rotation region may introduce co-flow of at least 1, 2, 3, 4, 5, or more buffers (e.g., particle-free, or containing one or more particles, such as polymeric or magnetic particles) to impart rotation on one or more cells within the channel. In some cases, a cell rotation region may introduce co-flow of at most 5, 4, 3, 2, or 1 buffer to impart the rotation of one or more cells within the channel. In some examples, the plurality of buffers may be co-flown at a same position along the length of the cell rotation region, or sequentially at different positions along the length of the cell rotation region. In some examples, the plurality of buffers may be the same or different. In several embodiments, the cell rotation region of the microfluidic channel is fabricated using a two-layer fabrication process so that the axis of rotation is perpendicular to the axis of cell downstream migration and parallel to cell lateral migration.

Cells may be imaged in at least a portion of the cell rotating region, while the cells are tumbling and/or rotating as they migrate downstream. Alternatively or in addition to, the cells may be imaged in an imaging region that is adjacent to or downstream of the cell rotating region. In some examples, the cells may be flowing in a single streamline within a flow channel, and the cells may be imaged as the cells are rotating within the single streamline. A rotational speed of the cells may be constant or varied along the length of the imaging region. This may allow for the imaging of a cell at different angles (e.g., from a plurality of images of the cell taken from a plurality of angles due to rotation of the cell), which may provide more accurate information concerning cellular features than can be captured in a single image or a sequence of images of a cell that is not rotating to any significant extent. This also allow a 3D reconstruction of the cell using available software since the angles of rotation across the images are known. Alternatively, every single image of the sequence of image many be analyzed individually to analyze (e.g., classify) the cell from each image. In some cases, results of the individual analysis of the sequence of images may be aggregated to determine a final decision (e.g., classification of the cell).

In some embodiments, a cell rotation region of a microfluidic channel incorporates an injected co-flow prior to an imaging region in accordance with an embodiment of the disclosure. Co-flow may be introduced in the z plane (perpendicular to the imaging plane) to spin the cells. Since the imaging is done in the x-y plane, rotation of cells around an axis parallel to the y-axis provides additional information by rotating portions of the cell that may have been occluded in previous images into view in each subsequent image. Due to a change in channel dimensions, at point xo, a velocity gradient is applied across the cells, which can cause the cells to spin. The angular velocity of the cells depends on channel and cell dimensions and the ratio between Q1 (main channel flow rate) and Q2 (co-flow flow rate) and can be configured as appropriate to the requirements of a given application. In some embodiments, a cell rotation region incorporates an increase in one dimension of the microfluidic channel to initiate a change in the velocity gradient across a cell to impart rotation onto the cell. In some aspects, a cell rotation region of a microfluidic channel incorporates an increase in the z-axis dimension of the cross section of the microfluidic channel prior to an imaging region in accordance with an embodiment of the disclosure. The change in channel height can initiate a change in velocity gradient across the cell in the z axis of the microfluidic channel, which can cause the cells to rotate as with using co-flow.

### Flowing Cells

In some embodiments, the system and methods of the present disclosure focuses the cells in microfluidic channels. The term focusing as used herein broadly means controlling the trajectory of cell/cells movement and comprises controlling the position and/or speed at which the cells travel within the microfluidic channels. In some embodiments controlling the lateral position and/or the speed at which the particles travel inside the microfluidic channels, allows to accurately predict the time of arrival of the cell at a bifurcation. The cells may then be accurately sorted. The parameters critical to the focusing of cells within the microfluidic channels include, but are not limited to channel geometry, particle size, overall system throughput, sample concentration, imaging throughput, size of field of view, and method of sorting.

In some embodiments the focusing is achieved using inertial forces. In some embodiments, the system and methods of the present disclosure focus cells to a certain height from the bottom of the channel using inertial focusing. In these embodiments, the distance of the cells from the objective is equal and images of all the cells will be clear. As such, cellular details, such as nuclear shape, structure, and size appear clearly in the outputted images with minimal blur. In some aspects, the system disclosed herein has an imaging focusing plane that is adjustable. In some aspects, the focusing plane is adjusted by moving the objective or the stage. In some aspects, the best focusing plane is found by recording videos at different planes and the plane wherein the imaged cells have the highest Fourier magnitude, thus, the highest level of detail and highest resolution, is the best plane.

In some embodiments, the system and methods of the present disclosure utilize a hydrodynamic-based z focusing system to obtain a consistent z height for the cells of interests that are to be imaged. In some aspects, the design comprises hydrodynamic focusing using multiple inlets for main flow and side flow. In some aspects, the hydrodynamic-based z focusing system is a triple-punch design. In some aspects, the design comprises hydrodynamic focusing with three inlets, wherein the two side flows pinch cells at the center. For certain channel designs, dual z focus points may be created, wherein a double-punch design similar to the triple-punch design may be used to send objects to one of the two focus points to get consistent focused images. In some aspects, the design comprises hydrodynamic focusing with 2 inlets, wherein only one side flow channel is used and cells are focused near channel wall. In some aspects, the hydrodynamic focusing comprises side flows that do not contain any cells and a middle inlet that contains cells. The ratio of the flow rate on the side channel to the flow rate on the main channel determines the width of cell focusing region. In some aspects, the design is a combination of the above. In all aspects, the design is integrable with the bifurcation and sorting mechanisms disclosed herein. In some aspects, the hydrodynamic-based z focusing system is used in conjunction with inertia-based z focusing.

In some embodiments, the terms “particles”, “objects”, and “cells” are used interchangeably. In some aspects, the cell is a live cell. In some aspects, the cell is a fixed cell (e.g., in methanol or paraformaldehyde). In some cases, one or more cells may be coupled (e.g., attached covalently or non-covalently) to a substrate (e.g., a polymeric bead or a magnetic bead) while flowing through the flow cell. In some cases, the cell(s) may not be coupled to any substrate while flowing through the flow cell.

### Imaging and Classification

A variety of techniques can be utilized to classify images of cells captured by classification and/or sorting systems in accordance with various embodiments of the disclosure. In some embodiments, the image captures are saved for future analysis/classification either manually or by image analysis software. Any suitable image analysis software can be used for image analysis. In some embodiments, image analysis is performed using OpenCV. In some embodiments, analysis and classification is performed in real time.

In some embodiments, the system and methods of the present disclosure comprise collecting a plurality of images of objects in the flow. In some aspects, the plurality of images comprises at least 20 images of cells. In some aspects, the plurality of images comprises at least 19, 18, 17, 16, 15, 14, 13, 12, 11, 10, 9, 8, 7, 6, 5, 4, 3, or 2 images of cells. In some embodiments, the plurality of images comprises images from multiple cell angles. In some aspects, the plurality of images, comprising images from multiple cell angles, help derive extra features from the particle which would typically be hidden if the particle is imaged from a single point-of-view. In some aspects, without wishing to be bound by theory, the plurality of images, comprising images from multiple cell angles, help derive extra features from the particle which would typically be hidden if a plurality of images are combined into a multi-dimensional reconstruction (e.g., a two-dimensional hologram or a three-dimensional reconstruction).

In some embodiments, the systems and methods of present disclosure allow for a tracking ability, wherein the system and methods track a particle (e.g., cell) under the camera and maintain the knowledge of which frames belong to the same particle. In some embodiments, the particle is tracked until it has been classified and/or sorted. In some cases, the particle may be tracked by one or more morphological (e.g., shape, size, area, volume, texture, thickness, roundness, etc.) and/or optical (e.g., light emission, transmission, reflectance, absorbance, fluorescence, luminescence, etc.) characteristics of the particle. In some examples, each particle may be assigned a score (e.g., a characteristic score) based on the one or more morphological and/or optical characteristics, thereby to track and confirm the particle as the particle travels through the microfluidic channel.

In some embodiments, the systems and methods of the disclosure comprise imaging a single particle in a particular field of view of the camera. In some aspects, the system and methods of the present disclosure image multiple particles in the same field of view of camera. Imaging multiple particles in the same field of view of the camera can provide additional advantages, for example it will increase the throughput of the system by batching the data collection and transmission of multiple particles. In some instances, at least about 2, 3, 4, 5, 6, 7, 8, 9, 10, 20, 30, 40, 50, 60, 70, 80, 90, 100, or more particles are imaged in the same field of view of the camera. In some instances, 100 to 200 particles are imaged in the same field of view of the camera. In some instances, at most about 100, 90, 80, 70, 60, 50, 40, 30, 20, 10, 9, 8, 7, 6, 5, 4, 3, or 2 particles are imaged in the same field of view of the camera. In some cases, the number of the particles (e.g., cells) that are imaged in the same field of view may not be changed throughout the operation of the flow cell. Alternatively, the number of the particles (e.g., cells) that are imaged in the same field of view may be changed in real-time throughout the operation of the flow cell, e.g., to increase speed of the classification and/or sorting process without negatively affecting quality or accuracy of the classification and/or soring process.

The imaging region maybe downstream of the focusing region and the ordering region. Thus, the imaging region may not be part of the focusing region and the ordering region. In an example, the focusing region may not comprise or be operatively coupled to any imaging device that is configured to capture one or more images to be used for particle analysis (e.g., cell classification).

### Samples

In some embodiments, the particles (for e.g. cells) analyzed by the systems and methods disclosed herein are comprised in a sample. The sample may be a biological sample obtained from a subject. In some embodiments, the biological sample comprises a biopsy sample from a subject. In some embodiments, the biological sample comprises a tissue sample from a subject. In some embodiments, the biological sample comprises liquid biopsy from a subject. In some embodiments, the biological sample can be a solid biological sample, e.g., a tumor sample. In some embodiments, a sample from a subject can comprise at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or at least about 100% tumor cells from a tumor.

In some embodiments, the sample can be a liquid biological sample. In some embodiments, the liquid biological sample can be a blood sample (e.g., whole blood, plasma, or serum). A whole blood sample can be subjected to separation of cellular components (e.g., plasma, serum) and cellular components by use of a Ficoll reagent. In some embodiments, the liquid biological sample can be a urine sample. In some embodiments, the liquid biological sample can be a perilymph sample. In some embodiments, the liquid biological sample can be a fecal sample. In some embodiments, the liquid biological sample can be saliva. In some embodiments, the liquid biological sample can be semen. In some embodiments, the liquid biological sample can be amniotic fluid. In some embodiments, the liquid biological sample can be cerebrospinal fluid. In some embodiments, the liquid biological sample can be bile. In some embodiments, the liquid biological sample can be sweat. In some embodiments, the liquid biological sample can be tears. In some embodiments, the liquid biological sample can be sputum. In some embodiments, the liquid biological sample can be synovial fluid. In some embodiments, the liquid biological sample can be vomit.

In some embodiments, samples can be collected over a period of time and the samples may be compared to each other or with a standard sample using the systems and methods disclosed herein. In some embodiments the standard sample is a comparable sample obtained from a different subject, for example a different subject that is known to be healthy or a different subject that is known to be unhealthy. Samples can be collected over regular time intervals, or can be collected intermittently over irregular time intervals.

In some embodiments, the subject may be an animal (e.g., human, rat, pig, horse, cow, dog, mouse). In some instances, the subject is a human and the sample is a human sample. The sample may be a fetal human sample. The sample may be a placental sample (e.g., comprising placental cells). The sample may be from a multicellular tissue (e.g., an organ (e.g., brain, liver, lung, kidney, prostate, ovary, spleen, lymph node, thyroid, pancreas, heart, skeletal muscle, intestine, larynx, esophagus, and stomach), a blastocyst). The sample may be a cell from a cell culture. In some sample the subject is a pregnant human, or a human suspected to be pregnant.

The sample may comprise a plurality of cells. The sample may comprise a plurality of the same type of cell. The sample may comprise a plurality of different types of cells. The sample may comprise a plurality of cells at the same point in the cell cycle and/or differentiation pathway. The sample may comprise a plurality of cells at different points in the cell cycle and/or differentiation pathway.

The plurality of samples may comprise one or more malignant cell. The one or more malignant cells may be derived from a tumor, sarcoma, or leukemia.

The plurality of samples may comprise at least one bodily fluid. The bodily fluid may comprise blood, urine, lymphatic fluid, saliva. The plurality of samples may comprise at least one blood sample.

The plurality of samples may comprise at least one cell from one or more biological tissues. The one or more biological tissues may be a bone, heart, thymus, artery, blood vessel, lung, muscle, stomach, intestine, liver, pancreas, spleen, kidney, gall bladder, thyroid gland, adrenal gland, mammary gland, ovary, prostate gland, testicle, skin, adipose, eye or brain.

The biological tissue may comprise an infected tissue, diseased tissue, malignant tissue, calcified tissue or healthy tissue.

### Circulating Endometrial Cells

In some embodiments, the system and methods disclosed herein can be utilized to detect circulating endometrial cells, e.g., for non-invasive diagnosis of endometriosis as an alternative or additional approach to other surgical methods (e.g., visualization or biopsy under laparoscopy). Determination of a presence of one or more endometrial cells in circulation in a provided sample, their count, their isolation, and/or subsequent molecular analysis (e.g., for gene expression consistent with endometriosis) can help detection of endometriosis. Similar approaches can be utilized for detection/analysis of circulating endometrial cancer cells, e.g., for uterine/endometrial cancer detection.

### Circulating Endothelial Cells

In some embodiments, the system and methods disclosed herein can be utilized to detect circulating endothelial cells. The endothelium can be involved (e.g., directly involved) in diseases such as, e.g., peripheral vascular disease, stroke, heart disease, diabetes, insulin resistance, chronic kidney failure, tumor growth, metastasis, venous thrombosis, and severe viral infectious diseases. Thus, dysfunction of the vascular endothelium can be one of the hallmarks of human diseases (e.g., preeclampsia (a pregnancy specific disease), endocarditis, etc.). For example, detection of circulating endothelial cells can be utilized for detection of cardiovascular disease. Sorted endothelial cells can be further analyzed for molecular profiling, e.g., specific vascular endothelial cell RNA expression in the presence of various vascular disease states.

### Cancer Cells

Many cancers are diagnosed in later stages of the disease because of low sensitivity of existing diagnostic procedures and processes. More than 1.5 million people are diagnosed with cancer every year in the USA, of which 600,000 people die. Currently, the first cancer screening procedure involves the detection of a tumor. Many cancer tumors, such as breast cancer are detected by self-or clinical examination. However, these tumors are typically detected only after the tumor reach a volume of 1 mL or 1 cc, when it contains approximately 10⁹ cells. Routine screening by mammography is more sensitive and allows detection of a tumor before it becomes palpable, but only after they reach an inch in diameter. MRI, positron emission tomography (PET) and single-photon emission computed tomography (SPECT) can reveal even smaller tumors than can be detected by mammograms. However, these imaging methods present significant disadvantages. Contrast agents for magnetic resonance imaging (MRI) are toxic and radionuclides delivered for SPECT or PET examination are sources of ionizing radiation. Because of its relatively poor resolution, ovarian cancer often requires several follow up scans with computed tomography (CT) or MRI, while undertaking all precautions to protect possible pregnancies, to reveal fine anatomy of developing tumors. Additionally, all of these diagnostic techniques require dedicated facilities, expensive equipment, well trained staff, and financial coverages.

Cancer is commonly diagnosed in patients by obtaining a sample of the suspect tissue and examining the tissue under a microscope for the presence of malignant cells. While this process is relatively straightforward when the anatomic location of the suspect tissue is known, it can become quite challenging when there is no readily identifiable tumor or pre-cancerous lesion. For example, to detect the presence of lung cancer from a sputum sample requires one or more relatively rare cancer cells to be present in the sample. Therefore, patients having lung cancer may not be diagnosed properly if the sample does not perceptively and accurately reflect the conditions of the lung.

Conventional light microscopy, which utilizes cells mounted on glass slides, can only approximate 2D and 3D measurements because of limitations in focal plane depth, sampling angles, and problems with cell preparations that typically cause cells to overlap in the plane of the image. Another drawback of light microscopy is the inherent limitation of viewing through an objective lens where only the area within the narrow focal plane provides accurate data for analysis.

Flow cytometry methods generally overcome the cell overlap problem by causing cells to flow one-by-one in a fluid stream. Unfortunately, flow cytometry systems do not generate images of cells of the same quality as traditional light microscopy, and, in any case, the images are not three-dimensional.

In some embodiments, the system and methods disclosed herein enable the acquisition of three-dimensional imaging data of individual cells, wherein each individual cell from a cell population is imaged from a plurality of angles. In some aspects, the present disclosure is used to diagnose cancer, wherein individual cancer cells are identified, tracked, and grouped together. In some aspects, the cells are live.

In some embodiments, the system and methods disclosed herein are used for cancer diagnosis in a subject, the method comprising imaging a cell in a biological sample from the subject to collect a plurality of images of the cell and analyzing the plurality of images to determine if cancerous cells are present in the subject, wherein the cancerous cell is in a flow during imaging and is spinning, and wherein the plurality of images comprise images from a different spinning angles.

In some embodiments, the system and methods disclosed herein are used for cancer cell detection, wherein the cancerous cells are from biological samples and are detected and tracked as they pass through the system of the present disclosure.

In some embodiments, the system and methods disclosed herein are used to identify cancer cells from biological samples acquired from mammalian subjects, wherein the cell population is analyzed by nuclear detail, nuclear contour, presence or absence of nucleoli, quality of cytoplasm, quantity of cytoplasm, nuclear aspect ratio, cytoplasmic aspect ratio, or nuclear to cytoplasmic ratio. In some aspects, the cancer cells that are identified indicate the presence of cancer in the mammalian sample, including but not limited to, lymphoma, myeloma, neuroblastoma, breast cancer, ovarian cancer, lung cancer, rhabdomyosarcoma, small-cell lung tumors, primary brain tumors, stomach cancer, colon cancer, pancreatic cancer, urinary bladder cancer, testicular cancer, lymphomas, thyroid cancer, neuroblastoma, esophageal cancer, genitourinary tract cancer, cervical cancer, endometrial cancer, adrenal cortical cancer, or prostate cancer. In some aspects, the the cancer is metastatic cancer. In some aspects, the the cancer is an early stage cancer.

In some embodiments, the system and methods disclosed herein are used to image a large number of cells from a subject and collect a plurality of images of the cell, and to then classify the cells based on an analysis of one or more of the plurality of images; wherein the plurality of images comprise images from a plurality of cell angles and wherein the cell is tracked until the cell has been classified. In some aspects, the tracked cells are classified as cancerous. In some aspects, the subject is a human.

In some embodiments, the cells used in the methods disclosed herein are live cells. In some aspects, the cells that are classified as cancerous cells are isolated and subsequently cultured for potential drug compound screening, testing of a biologically active molecule, and/or further studies.

In some embodiments, the system and methods disclosed herein are used to identify cancer cells from a cell population from a mammalian subject. In some aspects, the subject is a human. In some aspects, the system and methods disclosed herein are used to determine the progression of a cancer, wherein samples from a subject are obtained from two different time points and compared using the methods of the present disclosure. In some aspects, the system and methods disclosed herein are used to determine the effectiveness of an anti-cancer treatment, wherein samples from a subject are obtained before and after anti-cancer treatment and comparing the two samples using the methods of the present disclosure.

In some embodiments, the system and methods disclosed herein comprise a cancer detection system that uses a rapidly trained neural network, wherein the neural network detects cancerous cells by analyzing raw images of the cell and provides imaging information from the pixels of the images to a neural network. In some aspects, the neural network performs recognition and identification of cancerous cells using information derived from an image of the cells, among others, the area, the average intensity, the shape, the texture, and the DNA (pgDNA) of the cells. In some aspects, the neural network performs recognition of cancerous cells using textural information derived from an image of the cells, among them angular second moment, contrast, coefficient of correlation, sum of squares, difference moment, inverse difference moment, sum average, sum variance, sum entropy, entry, difference variance, difference entropy, information measures, maximal correlation coefficient, coefficient of variation, peak transition probability, diagonal variance, diagonal moment, second diagonal moment, product moment, triangular symmetry and blobness.

Non-limiting examples of cancer of interest can include Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, and Wilms'tumor.

In some embodiments, the system and methods disclosed herein can detect and/or sort circulating tumor cells or liquid tumors. In cases where the primary tumor has been previously resected or inaccessible for other reasons, a biopsy of the main tissue may not be a viable option. As such, disseminated cancer cells can be found at a much lower concentration and purity in bodily fluids, such as circulating tumor cells (CTCs) in blood, peritoneal or pleural fluids, urine, etc.

### Immune Cells

In some embodiments, the system and methods disclosed herein can be utilized to isolate specific types or subtypes of immune cells. Examples of different types of immune cells can include, but are not limited to, neutrophils, eosinophils, basophils, mast cells, monocytes, macrophages, dendritic cells, natural killer (NK) cells, and lymphocytes (e.g., B cells, T cells). Additional examples of different types of immune cells can include, but are not limited to, native immune cells and engineered immune cells (e.g., engineered to express a heterologous cytokine, cytokine receptor, antigen, antigen receptor (e.g., chimeric antigen receptor or CAR), etc.). Examples of different sub-types of immune cells (e.g., T cells) can include, but are not limited to, naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. Additional examples of different sub-types of immune cells can include, but are not limited to, upregulation or downregulation of one or more of the following genes: CD3, CD4, CD8, CCR7, CD45RA, CD38, HLA, CD45RO, CCR4, CD24, CD127, CCR6, CXCR3, CD24, CD38, CD19, CD19, CD20, CD27, IgD, CD14, CD16, CD56, CD11c, and CD123. For example, T cells can comprise CD38+/HLA-DR+CD4+ activated T cells or CD38+/HLA-DR+/CD8+ activated T cells. In other examples, monocytes can comprise CD16+ non-classical monocytes or CD16-classical monocytes. In another example, dendritic cells can comprise CD11c+ myeloid dendritic cells or CD123+ plasmacytoid dendritic cells. In another example, NK cells can comprise CD16+ NK cells or CD16− NK cells. In some cases, an immune cell as disclosed herein may be characterized as an antibody producing cell.

In some embodiments, the system and methods disclosed herein can be utilized to isolate specific types or subtypes of T cells (e.g., CAR T cells) from a population of T cells. CAR T cells can be cells that have been genetically engineered to produce an artificial T-cell receptor for use in, e.g., immunotherapy. CAR T cells can be classified and sorted, using systems and methods disclosed herein, and further cultured and proliferated for the applications for, e.g., drug development.

### Liquid Biopsy

A liquid biopsy comprises the collection of blood and/or urine from a cancer patient with primary or recurrent disease and the analysis of cancer-associated biomarkers in the blood and/or urine. A liquid biopsy is a simple and non-invasive alternative to surgical biopsies that enables doctors to discover a range of information about a tumor. Liquid biopsies are increasingly being recognized as a viable, noninvasive method of monitoring a patient's disease progression, regression, recurrence, and/or response to treatment.

In some embodiments, the methods disclosed herein are used for liquid biopsy diagnostics, wherein the biopsy is a liquid biological sample that is passed through the system of the present disclosure. In some aspects, the liquid biological sample that is used for the liquid biopsy is less than 5 mL of liquid. In some aspects, the liquid biological sample that is used for the liquid biopsy is less than 4 mL of liquid. In some aspects, the liquid biological sample that is used for the liquid biopsy is less than 3 mL of liquid. In some aspects, the liquid biological sample that is used for the liquid biopsy is less than 2 mL of liquid. In some aspects, the liquid biological sample that is used for the liquid biopsy is less than 1 mL of liquid. In some aspects, the liquid biological sample that is used for liquid biopsy is centrifuged to get plasma.

In some embodiments, the system and methods of the present disclosure are used for body fluid sample assessment, wherein cells within a sample are imaged and analyzed and a report is generated comprising all the components within the sample, the existence of abnormalities in the sample, and a comparison to previously imaged or tested samples from the same patient or the baseline of other healthy individuals.

In some embodiments, the system and methods of the present disclosure are used for the diagnosis of immune diseases, including but not limited to tuberculosis (TB) and acquired immune deficiency disorder (AIDS), wherein white blood cells are imaged in the system disclosed herein to examine their capacity to release pro-and anti-inflammatory cytokines.

In some embodiments, the system and methods of the present disclosure are used to assess patient immune responses to immunomodulatory therapies by imaging their white blood cells and analyzing the change in their capacity to release pro-and anti-inflammatory cytokines.

In some embodiments, the system and methods of the present disclosure are used to identify the efficacy of therapeutics and/or to guide the selection of agents or their dosage by isolating patients'white blood cells and analyzing the effect of target therapeutics on their capacity to release pro-and anti-inflammatory cytokines.

In some embodiments, the system and methods of the present disclosure are used to isolate pure samples of stem cell-derived tissue cells by obtaining images of cells, and isolating cells with desired phenotype.

### Testing Biologically Active Molecules

In some embodiments, the methods disclosed herein are used for biologically active molecule testing, for example drugs. In some embodiments, the methods of the disclosure are sued to collect desired cells from a sample and then treating the desired cells with a biologically active molecule in order to test the effect of the biologically active molecule on the collected cells.

In some embodiments, the methods and systems of the present disclosure are used for identifying the efficacy of therapeutics. In some aspects, identifying the efficacy of therapeutics using the system disclosed herein is carried out by obtaining images of a cell before and after treatment and analyzing the images to determine whether the cell has responded to the therapeutic of interest.

In some embodiments, the system and methods disclosed herein are used for diseased cell detection, wherein the diseased cells are from biological samples and are detected and tracked as they pass through the system of the present disclosure. In some aspects, the diseased cells are isolated and grouped together for further studies.

In some embodiments, the cells used in the methods disclosed herein are live cells. In some aspects, the cells that are classified as diseased cells are isolated and subsequently cultured for potential drug compound screening, testing of a biologically active molecule, and/or further studies.

Although the present disclosure has been described in certain specific aspects, many additional modifications and variations would be apparent to those skilled in the art. It is therefore to be understood that the present disclosure can be practiced otherwise than specifically described without departing from the scope and spirit of the present disclosure. Thus, some embodiments of the present disclosure should be considered in all respects as illustrative and not restrictive.

### Point-of-Care Diagnostics

Any one of the systems and methods disclosed herein (e.g., cell morphology-based classification, such as for sorting or enrichment) can be utilized for point-of-care diagnostics. A point-of-care diagnostics or point-of-care diagnostics can encompass analysis of one or more samples (e.g., biopsy samples, such as blood samples) of a subject (e.g., a patient) in a point-of-care environment, such as, for example, hospitals, emergency departments, intensive care units, primary care setting, medical centers, patient homes, a physician's office, a pharmacy or a site of an emergency. The point-of-care diagnostics as disclosed herein can be utilized to identify a pathogen (e.g., any infectious agents, gems, bacteria, virus, etc.), identify immune response in the subject (e.g., via classifying and/or sorting specific immune cell types), generate a count of cells of interest (e.g., diseased cells, healthy cells, etc.), etc.

### IV. Computer Systems

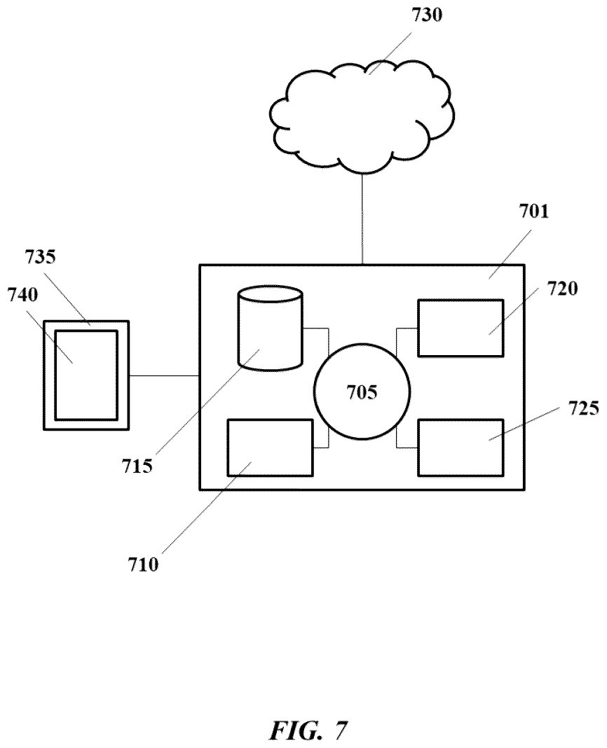

The present disclosure provides computer systems that are programmed to implement methods of the disclosure. FIG. 7 shows a computer system 701 that is programmed or otherwise configured to capture and/or analyze one or more images of the cell. The computer system 701 can regulate various aspects of components of the cell sorting system (or cell partitioning system) of the present disclosure, such as, for example, the pump, the valve, and the imaging device. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing, for example, the one or more images of the cell that is transported through the channel of the cell sorting system. In some cases, the computer system 701 can be configured to provide a live feedback of the images. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can be, for example, a deep learning algorithm to enable sorting of the cell.

### EXAMPLES

The following specific examples are illustrative and non-limiting. The examples described herein reference and provide non-limiting support to the various embodiments described in the preceding sections.

### Example 1. Label-Based Imaging and Label-Free Imaging

In some embodiments, the systems, methods, and compositions of the present disclosure can be utilized to (a) stain cells with multiple fluorescent markers, (b) image both label-free images (e.g., brightfield images) and fluorescent images, and (c) use fluorescent markers to aid cell typing and annotation. This can be useful for, e.g., (i) immunotyping, (ii) analysis and sorting of cells with cell states that are in continuum (e.g., non-binary cell states), such as T cell differentiation (e.g., IFN gamma +/−) or cancer epithelial-mesenchymal transition (EMT) (e.g., EpCAM +/−), and/or (iii) enhanced sorting purity (e.g., CD4+ T cells from tumor microenvironment).

### Example 2. Bulk Sorting and Downstream Single Cell Analysis

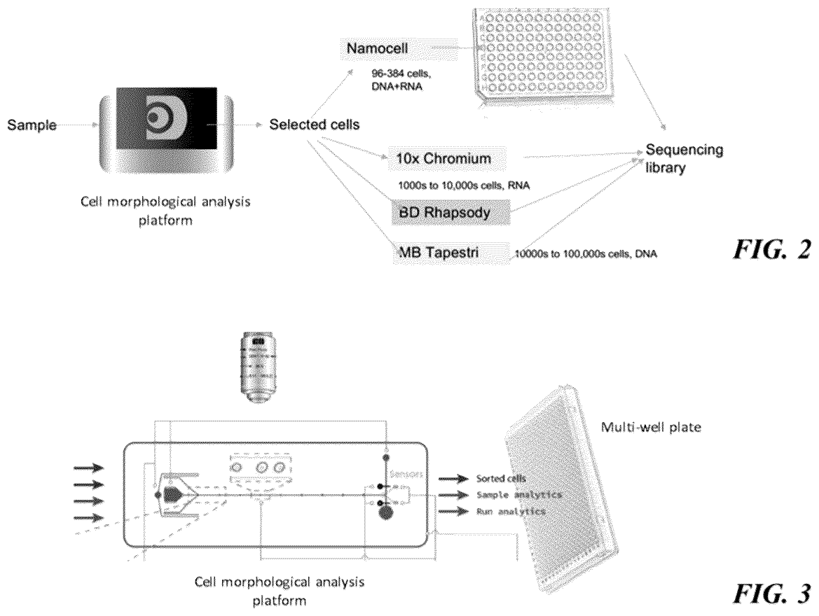

In some embodiments, the systems, methods, and compositions of the present disclosure can be utilized to (a) sort (or partition) a population of cells into two or more subpopulations in bulk (e.g., via morphological analysis of the population of cells), and (b) direct (e.g., automatically direct) the sorted (or partitioned) cells or fragments thereof for downstream single cell analysis. For example, as schematically illustrated in FIG. 2, the cell morphological analysis platform (e.g., cell analysis platform 500 as shown in FIG. 5) can partition one or more cells of interest from a population of cells, and direct the partitioned cells to a single cell analysis module (e.g., Nanocell, 10× Chromium, BD Rhapsody, MB Tapestri, etc.), which single cell module can be utilized to subject one or more components of each single cell (e.g., RNA, DNA) to further analysis (e.g., sequencing).

### Example 3. Single Cell Sorting Into Multi-Wells

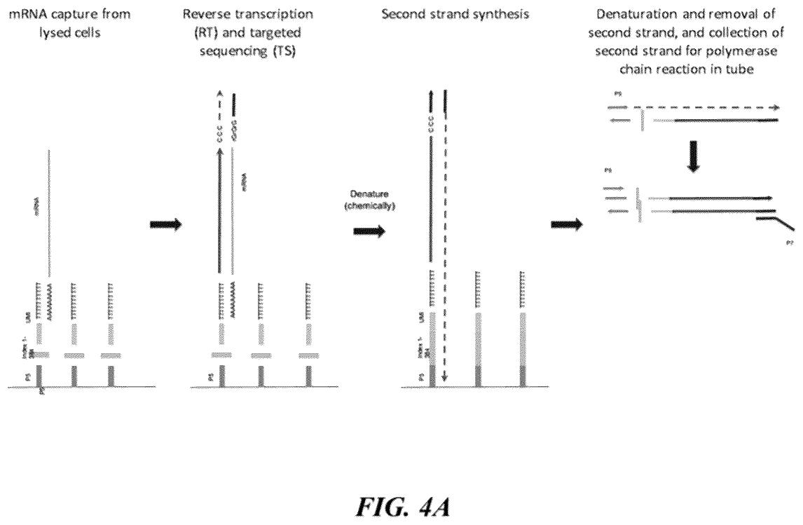

In some embodiments, the systems, methods, and compositions of the present disclosure can be utilized to (a) classify (e.g., sort in silico) a population of cells into two or more subpopulations in bulk (e.g., via morphological analysis of the population of cells), and (b) dispense the sorted cells into a multi-well plate (e.g., dispense each sorted or partitioned cell into a well of the multi-well plate). For example, as schematically illustrated in FIG. 3, the cell morphological analysis platform can be configured to (or operatively coupled to another module that is configured to) dispense each single cell of the sorted cells into a well of a multi-well plate or into a spot of a plurality of spots (e.g., via one or more nozzles). This can be useful for, e.g., (i) sorting single cell from each cluster of cells (e.g., cells from tumor microenvironment) and verify cell type and/or cell, (ii) identification of rare target cells, such as circulating tumor cells (CTCs), minimal residual disease (MRD) cells, etc., (iii) single cell functional analysis of live single cells, (iv) analysis of nucleic acid analytes (e.g., DNA, RNA) from single cells, (v) immunotyping (e.g., profiling and verifying a plurality of cell types in mixed samples), and (vi) tissue profiling or generating a cell atlas (e.g., correlating cell clustering map to single cell molecular analysis data).

In some embodiments, the multi-well plate or the plurality of spots can be functionalized with heterologous labels (e.g., nucleic acid barcodes) for sequencing (e.g., single cell RNA sequencing). For the case of the multi-well plate, the output wells can be filled with oil to generate droplets upon directing single cells into the oil suspension or, alternatively, the cell flow system can use pinching mechanism to generate droplets and direct the droplets into the wells. In some cases, N number (e.g., 384) of barcoding reagents can be used along with M number (e.g., 24) of output wells (or spots), thereby providing N×M (e.g., 9216) unique barcodes.

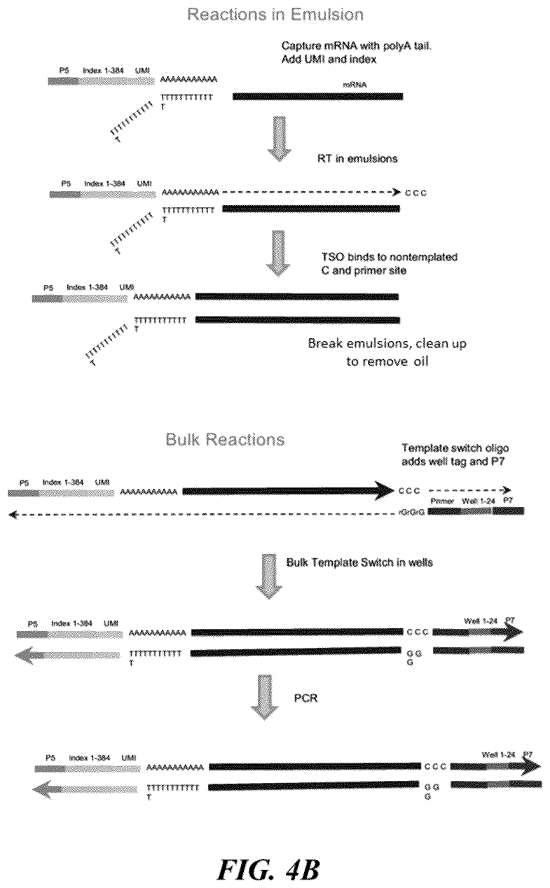

FIG. 4A schematically illustrates a spot of a plurality of spots on a slide (e.g., a microarray), wherein the spot is pre-coated with a nucleic acid barcode (or a barcode oligo) to capture a target mRNA from a partitioned single cell that is lysed, to subsequently generate one or more copies (e.g., amplification) of the target mRNA for sequencing.

FIG. 4B schematically illustrates a process of lysing a cell (e.g., a partitioned single cell as disclosed herein) and performing reverse transcription (RT) of a target mRNA from the lysed cell in an emulsion. In the emulsion, a template switch oligo (TSO) can hybridize to untemplated C nucleotides added by the reverse transcriptase during reverse transcription. Subsequently, the emulsions can be broken up and cleaned up (e.g., to remove oil), and the RT products can be utilized for bulk reactions (e.g., amplification for sequencing).

### EMBODIMENTS

The following non-limiting embodiments provide illustrative examples of the invention, but do not limit the scope of the invention.

Embodiment 1. A method of imaging a cell, the method comprising: staining the cell using at least one dye; rotating the cell in a field of view of an imaging device, and imaging the cell to create a cell image,
optionally wherein:
(1) the method further comprises delivering the cell to a partition, and correlating the partition to the image, further optionally wherein:
   (a) the partition comprises a well, optionally wherein only a single cell is delivered to the well; and/or
   (b) the partition comprises an aqueous droplet, further optionally wherein the aqueous droplet is suspended in a nonaqueous carrier; and/or
(2) the at least one dye comprises a nucleic acid staining dye; and/or
(3) the at least one dye comprises a chromatin staining dye; and/or
(4) the at least one dye comprises an organellar staining dye; and/or
(5) the at least one dye comprises a mitochondrial staining dye; and/or
(6) the at least one dye comprises a nuclear staining dye; and/or
(7) the at least one dye comprises a nucleolar staining dye; and/or
(8) the at least one dye comprises a cytoplasmic staining dye; and/or
(9) the at least one dye comprises a cell surface staining dye; and/or
(10) the at least one dye comprises cell surface protein staining dye; and/or
(11) the at least one dye comprises a fluorescent label bound to an epitope binding domain; and/or
(12) the at least one dye comprises a fluorescently labeled antibody; and/or
(13) rotating the cell comprises flowing the cell in a channel comprising fluids flowing at two flow rates; and/or
(14) the imaging the cell comprises (i) detecting the at least one dye coupled to the cell and (ii) subjecting the cell to label-free imaging, further optionally wherein:
   (a) the label-free imaging is brightfield imaging; and/or
   (b) (i) is performed prior to (ii) ; and/or
   (c) (i) is performed simultaneously with (ii) ; and/or
   (d) (i) is performed subsequent to (ii) ; and/or
   (e) the method further comprises (a) obtaining an imaging data based on (i) and an additional imaging data based on (ii), and (b) analyzing the imaging data based on analysis of the additional imaging data; and/or
   (f) the method further comprises (a) obtaining an imaging data based on (i) and an additional imaging data based on (ii), and (b) analyzing the additional imaging data based on analysis of the imaging data; and/or
   (g) the method further comprises plotting a cell clustering map based on (i) and (ii), further optionally wherein the cell clustering map is based on a cell morphology map.

Embodiment 2. A method comprising: contacting a first cell population to a dye that distinguishes a dye target feature of a subset of the first cell population; imaging the first cell population; identifying an image characteristic of the first population that correlates to dye binding; imaging a second cell population; and sorting the second cell population based upon presence of the image characteristic,
optionally wherein:
(1) the dye selectively binds to a feature of a subset of the population, further optionally wherein the dye target feature comprises a cell surface protein; and/or
(2) the dye target feature comprises cell size; and/or
(3) the dye target feature comprises cell shape; and/or
(4) the dye target feature comprises cell nucleus size; and/or
(5) the dye target feature comprises cell nucleus shape; and/or
(6) the dye target feature comprises cell surface topology; and/or
(7) the dye target feature comprises a cytoplasmic feature; and/or
(8) the dye target feature comprises a nucleolus; and/or
(9) the dye target feature comprises a cytoplasmic organelle; and/or
(10) the image characteristic comprises cell size; and/or
(11) the image characteristic comprises cell shape; and/or
(12) the image characteristic comprises cell nucleus size; and/or
(13) the image characteristic comprises cell nucleus shape; and/or
(14) the image characteristic comprises cell surface topology; and/or
(15) the image characteristic comprises a cytoplasmic feature
(16) the image characteristic is determined using machine learning; and/or
(17) the image characteristic is determined using an artificial intelligence algorithm; and/or
(18) the first cell population and the second cell population are drawn from a common source; and/or
(19) the first cell population and the second cell population are drawn from distinct sources; and/or
(20) sorting the second population comprises successively imaging a linear file of cells of the second population, and differentially depositing cells of the individual file of cells based upon presence of the image feature,
   optionally wherein differentially depositing cells comprises depositing cells to different reservoirs based upon presence of the image feature, further optionally wherein:
      (a) the reservoirs comprise wells; and/or
      (b) the method comprises correlating the reservoirs to status of the image feature in cells deposited to the reservoirs; and/or
(21) the method further comprises, prior to the imaging of the second cell population, contacting the second cell population to the dye, wherein the imaging of the second cell population comprises detecting the dye that is associated with the second cell population; and/or
(22) during the imaging of the second cell population, the second cell population is substantially free of the dye, wherein the imaging of the second cell population is label-free imaging; and/or
(23) the first cell comprises at least 2 cells; and/or
(24) the first cell comprises at least 5 cells; and/or
(25) the first cell comprises at least 10 cells; and/or
(26) the first cell comprises at least 20 cells; and/or
(27) the first cell comprises at least 50 cells; and/or
(28) the first cell comprises at most 2 cells; and/or
(29) the first cell comprises at most 5 cells; and/or
(30) the first cell comprises at most 10 cells; and/or
(31) the first cell comprises at most 20 cells; and/or
(32) the first cell comprises at most 50 cells.

Embodiment 3. A method of producing a population enriched for imaged cells sharing a common image characteristic, the method comprising: imaging a cell to obtain a cell image; comparing the cell image to a database; delivering the cell to a partition comprising a plurality of cells at least some of which have an image characteristic similar to an independent image of the database; and correlating the partition to the cell image,
optionally wherein:
(1) the plurality of cells of the partition are imaged prior to deposition in the partition; and/or
(2) the partition is a well, optionally wherein only a single cell is delivered to the well; and/or
(3) the partition comprises an aqueous droplet; and/or
(4) the partition harbors an unordered emulsion; and/or
(5) the image characteristic comprises an image feature; and/or
(6) the image characteristic is identified using machine learning; and/or
(7) the image characteristic is identified using an artificial intelligence algorithm; and/or
(8) the partition comprises no more than 50% of cells that do not have an image characteristic similar to the independent image of the cell database; and/or
(9) the partition comprises no more than 40% of cells that do not have an image characteristic similar to the independent image of the cell database; and/or
(10) the partition comprises no more than 30% of cells that do not have an image characteristic similar to the independent image of the cell database; and/or
(11) the partition comprises no more than 20% of cells that do not have an image characteristic similar to the independent image of the cell database; and/or
(12) the partition comprises no more than 10% of cells that do not have an image characteristic similar to the independent image of the cell database; and/or
(13) the partition comprises no more than 5% of cells that do not have an image characteristic similar to the independent image of the cell database; and/or
(14) the partition comprises no more than 1% of cells that do not have an image characteristic similar to the independent image of the cell database; and/or
(15) the partition comprises a heterologous marker, further optionally wherein:
   (a) the heterologous marker comprises a dye; and/or
   (b) the heterologous marker comprises a barcode; and/or
(16) the imaging of the cell comprises label-free imaging, further optionally wherein the label-free imaging is brightfield imaging; and/or
(17) the method further comprises, subsequent to the delivering, lysing the cell.

Embodiment 4. A method of cell sorting, the method comprising: generating a first image of a first cell of a population of cells; delivering the first cell to a first partition; generating a second image of a second cell of the population of cells, wherein the second image is similar to the first image; and delivering the second cell to the first partition,
optionally wherein:
(1) the first partition is a well, optionally wherein only a single cell is delivered to the well; and/or
(2) the first partition is an aqueous droplet; and/or
(3) correlating the first partition to the first image; and/or
(4) being similar comprises sharing an image feature; and/or
(5) being similar comprises a machine learning determination; and/or
(6) being similar comprises an artificial intelligence algorithm determination; and/or
(7) the first partition comprises no more than 50% of cells that do not have an image similar to the independent image of the cell database; and/or
(8) the first partition comprises no more than 40% of cells that do not have an image similar to the independent image of the cell database; and/or
(9) the first partition comprises no more than 30% of cells that do not have an image similar to the independent image of the cell database; and/or
(10) the first partition comprises no more than 20% of cells that do not have an image similar to the independent image of the cell database; and/or
(11) the first partition comprises no more than 10% of cells that do not have an image similar to the independent image of the cell database; and/or
(12) the first partition comprises no more than 5% of cells that do not have an image similar to the independent image of the cell database; and/or
(13) the first partition comprises no more than 1% of cells that do not have an image similar to the independent image of the cell database; and/or
(14) the method comprises generating a third image of a third cell of the population, wherein the third image is dissimilar from the first image, and delivering the third cell to a second partition, further optionally wherein:
   (a) the method comprises correlating the third image to the second partition; and/or
   (b) a degree of correlation factor between the first image and the third image is at most about 60%; and/or
   (c) a degree of correlation factor between the first image and the third image is at most about 50%; and/or
   (d) a degree of correlation factor between the first image and the third image is at most about 40%; and/or
   (e) a degree of correlation factor between the first image and the third image is at most about 30%; and/or
   (f) a degree of correlation factor between the first image and the third image is at most about 20%; and/or
   (g) a degree of correlation between the first image and the third image is at most about 10%; and/or
   (h) the method comprises adding a heterologous marker to the first partition, further optionally wherein (i) the heterologous marker comprises a dye, and/or (ii) the heterologous marker comprises a barcode; and/or
(15) the method comprises adding a heterologous marker to the second partition, further optionally wherein:
   (a) the heterologous marker comprises a dye; and/or
   (b) the heterologous marker comprises a barcode; and/or
(16) a degree of correlation factor between the first image and the second image is at least about 50%; and/or
(17) a degree of correlation factor between the first image and the second image is at least about 60%; and/or
(18) a degree of correlation factor between the first image and the second image is at least about 70%; and/or
(19) a degree of correlation factor between the first image and the second image is at least about 80%; and/or
(20) a degree of correlation factor between the first image and the second image is at least about 90%; and/or
(21) a degree of correlation factor between the first image and the second image is at least about 95%; and/or
(22) a degree of correlation factor between the first image and the second image is at least about 99%; and/or
(23) the method further comprises, subsequent to the delivering, lysing the first cell or the second cell.

Embodiment 5. A method of tracking an imaged cell, the method comprising: imaging a cell to generate an imaged cell; delivering the imaged cell to a partition; and associating an image of the imaged cell with the partition,
optionally wherein:
(1) the method comprises delivering a heterologous marker to the partition, further optionally wherein (A) the heterologous marker comprises a dye and/or (B) the heterologous marker comprises a barcode; and/or
(2) the partition is a well, optionally wherein only a single cell is delivered to the well; and/or (3) the partition comprises an oil; and/or
(4) the partition comprises at least one cell processing reagent, further optionally wherein:
   (a) the processing reagent comprises a cell fixative; and/or
   (b) the processing reagent comprises a cell lysis reagent; and/or
   (c) the processing reagent comprises a reverse transcriptase; and/or
   (d) the processing reagent comprises a cell stain; and/or
   (e) the processing reagent comprises a nucleic acid guided endonuclease; and/or
   (f) the processing reagent comprises a dye; and/or
   (g) the processing reagent comprises a pharmaceutical, further optionally wherein:
      (A) the pharmaceutical comprises a cell division inhibitor; and/or
      (B) the pharmaceutical comprises a cell growth inhibitor; and/or
      (C) the pharmaceutical comprises a cell differentiation inhibitor; and/or
(5) the partition comprises a plurality of cells having dissimilar images
(6) the partition comprises a plurality of cells sharing a common machine learning categorization; and/or
(7) the partition comprises a plurality of cells sharing a common artificial intelligence algorithm categorization; and/or
(8) the partition comprises a plurality of cells having similar images,
   optionally wherein a majority of the cells of the partition have similar images, further optionally wherein:
      (A) the majority comprises at least 50% of the cells of the partition; and/or
      (B) the majority comprises at least 90% of the cells of the partition; and/or
      (C) the majority comprises at least 95% of the cells of the partition; and/or
      (D) the majority comprises at least 99% of the cells of the partition; and/or
(9) delivering the imaged cell to the partition comprises associating the imaged cell with a tag, and co-delivering the imaged cell and the tag to the partition, further optionally wherein:
   (a) the tag does not associate with the plurality of cells having dissimilar images; and/or
   (b) the tag identifies nucleic acids of the imaged cell; and/or
   (c) co-delivering the imaged cell and the tag to the partition comprises colocalizing the cell and the tag to a common aqueous droplet, and depositing the aqueous droplet to an oil carrier in the partition; and/or
(10) the image is generated via the imaging of the cell; and/or
(11) the image is a label-free image; and/or
(12) the image is a bright-field image; and/or
(13) the method further comprises lysing the imaged cell at the partition; and/or
(14) the method further comprises lysing the imaged cell prior to or during delivery towards the partition.

Embodiment 6. A reservoir comprising a plurality of individually imaged cells, wherein an individually imaged cell of the plurality is labeled with a heterologous marker,
optionally wherein:
(1) an extracellular portion of the individually imaged cell is labeled with the heterologous marker; and/or
(2) an intracellular portion of the individually imaged cell is labeled with the heterologous marker,
   optionally wherein a polynucleotide molecule derived from the individually imaged cell is labeled with the heterologous marker, further optionally wherein:
      (A) the polynucleotide molecule is a DNA molecule; and/or
      (B) the polynucleotide molecule an RNA molecule; and/or
      (C) the heterologous marker comprises a polynucleotide sequence exhibiting complementarity to at least a portion of the polynucleotide molecule; and/or
(3) the individually imaged cell is suspended in an individual droplet in an emulsion, and wherein the individual droplet is labeled with the heterologous marker, optionally wherein:
   (a) an image of the individually imaged cell is digitally labeled, such that the image is correlated with the individually imaged cell that is labeled with the heterologous marker; and/or
   (b) the individual droplets comprise at least one cell processing reagent, further optionally wherein:
      (A) the processing reagent comprises a cell fixative; and/or
      (B) the processing reagent comprises a cell lysis reagent; and/or
      (C) the processing reagent comprises a reverse transcriptase; and/or
      (D) the processing reagent comprises a cell stain; and/or
      (E) the processing reagent comprises a nucleic acid guided endonuclease; and/or
      (F) the processing reagent comprises a dye; and/or
      (G) the processing reagent comprises a pharmaceutical, further optionally wherein (i) the pharmaceutical comprises a cell division inhibitor, and/or (ii) the pharmaceutical comprises a cell growth inhibitor, and/or (iii) the pharmaceutical comprises a cell differentiation inhibitor; and/or
(4) the heterologous maker comprises a barcode; and/or
(5) the heterologous marker comprises an antibody exhibiting specific binding to at least the portion of the cell, or an antigen-binding fragment thereof; and/or
(6) the plurality comprises at least 5 cells; and/or
(7) the plurality comprises at least 100 cells; and/or
(8) the plurality comprises at least 1,000 cells; and/or
(9) the plurality comprises at least 10,000 cells; and/or
(10) the plurality comprises at least 100,000 cells; and/or
(11) the reservoir comprises a well, optionally wherein:
   (a) a majority of the individually imaged cells correspond to images that belong to a common class, further optionally wherein:
      (A) the common class is defined by a cell structural feature; and/or
      (B) the common class is defined by a machine learning determination; and/or
      (C) the common class is defined by an artificial intelligence algorithm; and/or
      (D) the majority comprises at least 50% of the cells of the partition; and/or
      (E) the majority comprises at least 90% of the cells of the partition; and/or
      (F) the majority comprises at least 95% of the cells of the partition; and/or
      (G) the majority comprises at least 99% of the cells of the partition; and/or
   (b) cells of the plurality of individually imaged cells do not share a common image class corresponding to greater than 50% of the plurality of individually imaged cells; and/or
   (c) only a single cell is delivered to the well.

Embodiment 7. An emulsion comprising a plurality of aqueous partitions in an oil carrier held in a single well, wherein at least some of the aqueous partitions comprise one individually imaged cell per partition,
optionally wherein:
(1) the one individually imaged cell per partition is imaged ahead of being deposited into the partition; and/or
(2) a majority of the individually imaged cells map to images of a common class; and/or
(3) a majority of the individually imaged cells do not map to images of a common class; and/or
(4) at least some of the aqueous partitions comprising one individually imaged cell per partition also comprise a partition-identifying marker, optionally wherein:
   (a) the marker comprises a dye; and/or
   (b) the marker comprises a well number; and/or
   (c) the marker comprises a bar code, further optionally wherein:
      (A) the bar code comprises an oligonucleotide; and/or
      (B) the bar code identifies an aqueous partition; and/or
      (C) the bar code identifies an image of a common class; and/or
      (D) the bar code identifies a cell of an aqueous partition; and/or
      (E) the bar code identifies a polynucleotide sequence derived from an individually imaged cell of a partition of the plurality of aqueous partitions, further optionally wherein (i) the polynucleotide sequence is DNA, and/or (ii) the polynucleotide sequence is RNA; and/or
   (d) the partition-identifying marker correlates to an image of an individually imaged cell.

Embodiment 8. A method of tracking an imaged cell, the method comprising: imaging a cell; and delivering the imaged cell and a marker to a common partition,
optionally wherein:
(1) the marker comprises a barcode oligo; and/or
(2) the marker comprises an oligo-tagged antibody; and/or
(3) the marker comprises an oligo-tagged binding moiety; and/or
(4) the marker comprises a dye; and/or
(5) the marker comprises a fluorophore; and/or
(6) the method comprises recording the barcode oligo with which the imaged cell is delivered so as to associate the image to the barcode, optionally wherein:
   (a) the partition is a well, optionally wherein only a single cell is delivered to the well; and/or
   (b) the partition comprises a droplet in an oil; and/or
   (c) the partition comprises a spot on a surface, further optionally wherein (i) the spot comprises a plurality of oligos and/or (ii) the spot comprises a dye; and/or
   (d) the partition comprises at least one cell processing reagent, further optionally wherein:
      (A) the processing reagent comprises a cell fixative; and/or
      (B) the processing reagent comprises a cell lysis reagent; and/or
      (C) the processing reagent comprises a reverse transcriptase; and/or
      (D) the processing reagent comprises a cell stain; and/or
      (E) the processing reagent comprises a nucleic acid guided endonuclease; and/or
      (F) the processing reagent comprises a dye; and/or
      (G) the processing reagent comprises a pharmaceutical, optionally wherein: (i) the pharmaceutical comprises a cell division inhibitor, and/or (ii) the pharmaceutical comprises a cell growth inhibitor, and/or (iii) the pharmaceutical comprises a cell differentiation inhibitor.

Embodiment 9. A method of tracking an imaged cell, the method comprising: providing surface comprising a plurality of marked spots; imaging a cell; and delivering the imaged cell to a marked spot of the plurality of barcode oligo spots,
optionally wherein:
(1) the marked spots comprise dyes; and/or
(2) the marked spots comprise barcode oligos, optionally wherein delivering the imaged cell to a barcode oligo spot of the plurality of barcode oligo spots comprises recording the barcode oligo spot to which the imaged cell is delivered so as to associate the imaged cell to the barcode oligo spot; and/or
(3) delivering the imaged cell to a barcode oligo spot of the plurality of barcode oligo spots comprises associating an image of the imaged cell with the barcode oligo spot; and/or
(4) the method comprises imaging a second cell, grouping the second cell and the imaged cell into distinct classes, and delivering the second cell to the barcode oligo spot of the first cell; and/or
(5) the method comprises imaging a second cell, grouping the second cell and the imaged cell into a common class, and delivering the second cell to the barcode oligo spot of the first cell; and/or
(6) the barcode oligo spot comprises a second cell sharing a common image with the imaged cell; and/or
(7) the barcode oligo spot comprises a population of cells sharing images similar to the imaged cell, further optionally wherein:
   (a) the population comprises at least 5 cells; and/or
   (b) the population comprises at least 100 cells; and/or
   (c) the population comprises at least 1,000 cells; and/or
   (d) the population comprises at least 10,000 cells; and/or
   (e) the population comprises at least 100,000 cells; and/or
   (f) the reservoir comprises a well, optionally wherein only a single cell is delivered to the well; and/or
   (g) the majority comprises at least 50% of the cells of the partition; and/or
   (h) the majority comprises at least 90% of the cells of the partition; and/or
   (i) the majority comprises at least 95% of the cells of the partition; and/or
   (j) the majority comprises at least 99% of the cells of the partition; and/or
   (k) cells of the plurality of individually imaged cells do not share a common image class corresponding to greater than 50% of the plurality of individually imaged cells; and/or
(8) the barcode oligo spot comprises a cell processing reagent, further optionally wherein:
   (a) the processing reagent comprises a cell fixative; and/or
   (b) the processing reagent comprises a cell lysis reagent; and/or
   (c) the processing reagent comprises a reverse transcriptase; and/or
   (d) the processing reagent comprises a cell stain; and/or
   (e) the processing reagent comprises a nucleic acid guided endonuclease; and/or
   (f) the processing reagent comprises a dye; and/or
   (g) the processing reagent comprises a pharmaceutical, further optionally wherein (A) the pharmaceutical comprises a cell division inhibitor, and/or (B) the pharmaceutical comprises a cell growth inhibitor, and/or (C) the pharmaceutical comprises a cell differentiation inhibitor; and/or
(9) the method comprises delivering a cell processing reagent to the barcode oligo spot.

Embodiment 10. A method of assigning characteristics to subpopulations of a cell population, the method comprising measuring a phenotypic characteristic of at least some cells of the cell population; assigning the at least some cells of the cell population to one subpopulation of at least two subpopulations; and correlating the one subpopulation of at least two subpopulations to an independently expected subpopulation,
optionally wherein:
(1) measuring a phenotypic characteristic comprises imaging at least some cells of the cell population; and/or
(2) assigning the at least some cells of the cell population to one subpopulation of at least two subpopulations comprises a machine learning assessment; and/or
(3) assigning the at least some cells of the cell population to one subpopulation of at least two subpopulations comprises using an artificial intelligence algorithm; and/or
   optionally wherein imaging at least some cells of the cell population comprises contacting the cat least some cells of the cell population to at least one dye, further optionally wherein:
      (a) the at least one dye comprises a nucleic acid staining dye; and/or
      (b) the at least one dye comprises a chromatin staining dye; and/or
      (c) the at least one dye comprises an organellar staining dye; and/or
      (d) the at least one dye comprises a mitochondrial staining dye; and/or
      (e) the at least one dye comprises a nuclear staining dye; and/or
      (f) the at least one dye comprises a nucleolar staining dye; and/or
      (g) the at least one dye comprises a cytoplasmic staining dye; and/or
      (h) the at least one dye comprises a cell surface staining dye; and/or
      (i) the at least one dye comprises cell surface protein staining dye; and/or
      (j) the at least one dye comprises a fluorescent label bound to an epitope binding domain; and/or
      (k) the at least one dye comprises a fluorescently labeled antibody; and/or
(4) assigning comprises delivering the at least some cells to a distinct partition. optionally wherein:
   (a) the distinct partition comprises a well, optionally wherein only a single cell is delivered to the well; and/or
   (b) the distinct partition comprises a bead; and/or
   (c) the distinct partition comprises a spot on a surface, further optionally wherein the spot comprises at least one barcode; and/or
(5) correlating comprises attributing at least one trait of the independently expected subpopulation to the at least some cells of the cell population,
   optionally wherein:
   (a) the at least one trait comprises presence of a surface protein; and/or
   (b) the at least one trait comprises a genetic allele; and/or
   (c) the at least one trait comprises a biochemical trait; and/or
   (d) the at least one trait comprises response to a pharmaceutical; and/or
   (e) the at least one trait comprises a transcriptome expression profile; and/or
   (f) the at least one trait comprises an mRNA expression level; and/or
   (g) the at least one trait comprises a proteome expression profile; and/or
   (h) the at least one trait comprises a protein expression level.

Embodiment 11. A method of attributing characteristics to subpopulations of a cell population, comprising assigning cells of a cell population into a plurality of subpopulations; correlating the plurality of subpopulations to a cell population dataset comprising dataset subpopulations having known characteristics, and attributing the known characteristics to subpopulations of the plurality of subpopulations,
optionally wherein:
(1) assigning comprises measuring a phenotypic characteristic of the cells of the cell population, and assigning the cells based upon the phenotypic characteristic; and/or
(2) measuring a phenotypic trait comprises imaging the cells,
   optionally wherein:
   (a) the imaging comprises contacting the cells to at least one dye, further optionally wherein:
      (A) the at least one dye comprises a nucleic acid staining dye; and/or
      (B) the at least one dye comprises a chromatin staining dye; and/or
      (C) the at least one dye comprises an organellar staining dye; and/or
      (D) the at least one dye comprises a mitochondrial staining dye; and/or
      (E) the at least one dye comprises a nuclear staining dye; and/or
      (F) the at least one dye comprises a nucleolar staining dye; and/or
      (G) the at least one dye comprises a cytoplasmic staining dye; and/or
      (H) the at least one dye comprises a cell surface staining dye; and/or
      (I) the at least one dye comprises cell surface protein staining dye; and/or
      (J) the at least one dye comprises a fluorescent label bound to an epitope binding domain; and/or
      (K) the at least one dye comprises a fluorescently labeled antibody; and/or
   (b) the imaging comprises dye-free imaging; and/or
(3) correlating comprises assessing relative cell numbers for the subpopulations; and/or optionally wherein the method comprises correlating relative cell numbers for the cell populations to relative cell numbers for the dataset; and/or
(4) correlating comprises matching the phenotypic characteristic of the cells to a characteristic of the cell population dataset; and/or
   optionally wherein correlating comprises attributing at least one trait of the dataset to the at least some cells of the cell population, further optionally wherein:
      (A) the at least one trait comprises presence of a surface protein; and/or
      (B) the at least one trait comprises a genetic allele; and/or
      (C) the at least one trait comprises a biochemical trait; and/or
      (D) the at least one trait comprises response to a pharmaceutical; and/or
      (E) the at least one trait comprises a transcriptome expression profile; and/or
      (F) the at least one trait comprises an mRNA expression level; and/or
      (G) the at least one trait comprises a proteome expression profile; and/or
      (H) the at least one trait comprises a protein expression level.

Other features of systems and methods for imaging, analyzing, and/or classifying cells may be described in, for example, Patent Cooperation Treaty Patent Application No. PCT/US2019/046557 (“Systems and methods for particle analysis”) and Patent Cooperation Treaty Patent Application No. PCT/US2022/016748 (“Systems and methods for cell analysis”), each of which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of imaging a cell, the method comprising:
staining the cell using at least one dye;
rotating the cell in a field of view of an imaging device, and
imaging the cell to create a cell image.

2. The method of claim 1, further comprising delivering the cell to a partition, and correlating the partition to the image.

3. The method of claim 2, wherein the partition comprises a well.

4. The method of claim 1, wherein the cell is a single cell.

5. The method of claim 1, wherein the at least one dye is selected from the group consisting of a nucleic acid staining dye, a chromatin staining dye, an organellar staining dye, a mitochondrial staining dye, a nuclear staining dye, a nucleolar staining dye, a cytoplasmic staining dye, a cell surface staining dye, a cell surface protein staining dye, a fluorescent label bound to an epitope binding domain, and a fluorescently labeled antibody.

6. The method of claim 5, wherein the at least one dye is a nucleic acid staining dye.

7. The method of claim 5, wherein the at least one dye is a fluorescently labeled antibody.

8. The method of claim 1, wherein rotating the cell comprises flowing the cell in a channel comprising fluids flowing at two flow rates.

9. The method of claim 1, wherein the imaging the cell comprises (i) detecting the at least one dye coupled to the cell and (ii) subjecting the cell to label-free imaging.

10. The method of claim 9, wherein the label-free imaging is brightfield imaging.

11. A method of producing a population enriched for imaged cells sharing a common image characteristic, the method comprising: imaging a cell to obtain a cell image; comparing the cell image to a database; delivering the cell to a partition comprising a plurality of cells at least some of which have an image characteristic similar to an independent image of the database; and correlating the partition to the cell image.

12. The method of claim 11, wherein the image characteristic is identified using machine learning.

13. The method of claim 11, wherein the partition comprises no more than 50%, no more than 20%, no more than 10%, no more than 5%, or no more than 1% of cells that do not have an image characteristic similar to the independent image of the cell database.

14. A method of tracking an imaged cell, the method comprising: imaging a cell to generate an imaged cell; delivering the imaged cell to a partition; and associating an image of the imaged cell with the partition.

15. The method of claim 14, further comprising delivering a heterologous marker to the partition.

16. The method of claim 15, wherein the heterologous marker comprises a dye.

17. The method of claim 15, wherein the heterologous marker comprises a barcode.

18. The method of claim 14, wherein the partition is a well.

19. The method of claim 14, wherein the cell is a single cell.

20. The method of claim 14, wherein the partition comprises at least one cell processing reagent.
